(19) United States
(12) Patent Application Publication
(10) Pub. No.: US 2025/0362189 A1
(43) Pub. Date: Nov. 27, 2025

(54) FORCE SENSING SENSOR

(71) Applicant: UCL BUSINESS LTD, London (GB)

(72) Inventor: Constantinos HERACLEOUS, Larnaca (CY)

(21) Appl. No.: 18/872,094

(22) PCT Filed: Jun. 8, 2023
(86) PCT No.: PCT/GB2023/051495

§ 371 Date: Dec. 5, 2024

(30) Foreign Application Priority Data
Jun. 9, 2022 (GB) 2208480.0

(51) Int. Cl. G01L 1/22

(52) U.S. Cl. CPC G01L 1/2287

(57) ABSTRACT

A sensor cell is provided for measuring force, comprising: a conductive elastic substrate (13); and a variable resistor comprising: a first electrode (11a); a second electrode (12); and a portion (130) of the conductive elastic substrate (13) arranged between the electrodes (11a, 12) such that a force acting on the substrate (13) causes a change in distance between the electrodes (11a, 12) thereby changing a resistance of the variable resistor.

### TECHNICAL FIELD

The present invention relates to a sensor cell for measuring shear force, a system for measuring shear force, and a method of manufacturing a sensor cell.

### BACKGROUND

Force sensing is utilised in many technical fields, for example in the fields of haptic feedback systems and robotics. Force sensing may be used to determine pressure and/or shear force. An example of a specific application of force sensing is in measuring pressure and shear force at the plantar surface of the human foot, for example in the field of rehabilitation and sports science.

Various sensing technologies for measuring pressure and/or shear force are known. Existing sensors are typically based on capacitive transduction or inductive transduction. There is a need for low cost force sensors, particularly those suitable for measurement of shear forces. Furthermore, it is desirable to improve the sensitivity and range of force sensors (including those used to measure shear forces).

### SUMMARY OF INVENTION

A first aspect of the invention provides a sensor cell for measuring force, comprising:
a conductive elastic substrate; and
a variable resistor comprising:
   a first electrode;
   a second electrode; and
   a portion of the conductive elastic substrate arranged between the electrodes such that a force acting on the substrate causes a change in distance between the electrodes to change a resistance of the variable resistor.

In use, when a voltage is applied across the electrodes, a change in distance between the electrodes may cause a change in the resistance of the variable resistor in accordance with the equation (1):

$$R = \frac{ρ L}{A} \qquad ( 1 )$$

where R is the resistance of the variable resistor, L is the distance between the electrodes, ρ is the resistivity of the conducting path between the electrodes through the substrate, and A is the cross-sectional area of the electrodes. The magnitude of the force acting on the substrate can be inferred from the change in resistance following suitable calibration of the sensor cell.

In addition to the change in resistance arising from geometric deformation of the conductive elastic substrate (defined in equation (1)), the elastic substrate may have a bulk resistivity that varies in response to strain arising from deformation of the substrate due to a change in the distance between the electrodes.

The sensor cell may be arranged to measure shear force and/or pressure. Pressure may be measured by determining the magnitude of a distributed normal load acting on the substrate, followed by determining pressure based on the known surface area of the substrate.

The sensor cell may comprise:
a plurality of variable resistors each comprising:
   a first electrode;
   a second electrode; and
   a portion of the conductive elastic substrate, wherein the portion of the substrate is arranged between the electrodes.

A shear force acting on the substrate may cause a distance between the electrodes of a first subset of the resistors to increase and a distance between the electrodes of a second subset of the resistors to decrease, and a pressure acting on the substrate may cause a distance between the electrodes of both subsets of resistors to decrease.

A shear force acting on the substrate may cause the resistance of the first subset of resistors to increase as the distance between the respective electrodes increases and the resistance of the second subset of resistors to decrease as the distance between the respective electrodes decreases. The direction of the shear force can be determined from which subset of the plurality of resistors has an increase in resistance and which subset has a decrease in resistance, the shear force acting towards the variable resistor(s) for which the distance between the respective electrodes decreases, i.e., for which the resistance decreases. The magnitude of the shear force can be determined from the difference between the resistances of the variable resistors. The magnitude of a pressure acting on the substrate can be determined from the sum of the resistances of the variable resistors.

Each of the first and second subsets of variable resistors may comprise two resistors, wherein:
a shear force acting on the substrate along a first axis in a first direction causes a distance between the electrodes of a first resistor of the first subset of resistors to decrease and a distance between the electrodes of a second resistor of the first subset of resistors to increase;
a shear force acting on the substrate along the first axis in a second direction, opposite the first direction, causes a distance between the electrodes of the first resistor of the first subset of resistors to increase and a distance between the electrodes of the second resistor of the first subset of resistors to decrease;
a shear force acting on the substrate along a second axis in a first direction causes a distance between the electrodes of a first resistor of the second subset of resistors to decrease and a distance between the electrodes of a second resistor of the second subset of resistors to increase; and
a shear force acting on the substrate along the second axis in a second direction opposite the first direction causes a distance between the electrodes of the first resistor of the second subset of resistors to increase and a distance between the electrodes of the second resistor of the second subset of resistors to decrease.

It will be apparent that the resistors of the first subset of resistors are spaced apart along the first axis and the resistors of the second subset of resistors are spaced apart along the second axis. The first and second axes may be arranged perpendicularly.

In this way, the sensor cell provides three-axis force sensing; shear force sensing along two axes, in two directions for each axis, as well as force sensing in the normal direction. Force sensing in the normal direction allows for pressure sensing, based on the magnitude of a distributed normal load and the known surface area of the sensor cell. The three force components can also be used to determine the total force vector acting on the sensor cell using known trigonometrical techniques.

In embodiments of the invention, the sensor cell may comprise any suitable number of variable resistors. For example, the sensor cell may comprise four sets of two variable resistors, with the variable resistors of each set spaced apart along a different axis. In the manner described above, this would enable shear force to be measured along four different axes.

The first electrodes of the variable resistors may be separate. The resistors may share a common second electrode. This advantageously reduces the number of parts required to assemble the sensor cell, thereby reducing the cost and complexity of the sensor cell.

The substrate may take any suitable form such that a shear force acting on the substrate causes a change in distance between the electrodes of the variable resistor(s), and optionally such that a pressure acting on the substrate causes a change in distance between the electrodes of each variable resistor. The substrate may comprise petals/lobes separated by slots. The substrate may be suspended by radial suspension arms between the petals that extend from an edge of the substrate to a central boss region of the substrate. A bump may be fixed to the central boss region. The substrate may be circular.

The substrate may comprise a first part and a second part, each part comprising:
a first portion and a second portion;
an inclined portion extending from the first portion to the second portion;
a pivot arranged on the first portion;
a slot extending inwards from an edge of the inclined portion and extending perpendicularly to the pivot; wherein
the first and second parts are connected together (or joined together) at the respective slots.

At least a part of the substrate may be additive manufactured. Any suitable method of additive manufacturing may be used. In some embodiments, at least a part of the substrate may be manufactured using fused filament fabrication (FFF).

The substrate may comprise a plurality of outer walls surrounding an infill volume. The volume fraction of solid material in the infill volume may define an infill percentage. The infill percentage may be at least 50%, or at least 75%.

At least a part of the substrate may comprise thermoplastic polyurethane loaded with carbon black. An example of such a material is NinjaTek® Eel®.

The sensor cell may comprise one or more bumps coupled to the substrate such that the or each bump exerts a force on the substrate when a corresponding force is exerted on the bump. The sensor cell may comprise a bump for each variable resistor.

The sensor cell may comprise an insulation layer. The first electrode of the or each variable resistor may be arranged between the insulation layer and the substrate.

Where the sensor cell comprises the bump, the insulation layer may be arranged between the bump and the first electrode of the or each variable resistor.

The sensor cell may comprise an insulation layer, wherein the second electrode of each variable resistor is arranged between the insulation layer and the substrate.

The sensor cell may comprise a spacer arranged between the substrate and the second electrode of the or each variable resistor. The spacer may comprise an open area to allow contact between the substrate and the respective electrode. The spacer May comprise a non-conductive material. The spacer may comprise polydimethylsiloxane (PDMS) or a non-conducting thermoplastic. The sensor cell may comprise a packaging frame that is co-formed with the spacer.

The spacer may advantageously reduce hysteresis in measurements obtained using the sensor cell by causing the sensor cell to return to an open circuit after forces exerted on the substrate are removed. In some embodiments the spacer may be omitted. In a device with no spacer, the conductive elastic substrate may be in contact with the first electrode and the second electrode when not under load.

A second aspect of the invention provides a sensor array comprising a plurality of sensor cells of the first aspect of the invention (including any optional features thereof).

A third aspect of the invention provides a system for measuring force, comprising:
the sensor cell of the first aspect of the invention or the sensor array of the second aspect of the invention;
a power source arranged to provide a potential difference between the first electrode and the second electrode of each variable resistor;
one or more measuring devices configured to measure a change in an electrical property resulting from a change of resistance of each variable resistor caused by a force acting on the substrate; and
a processor configured to determine the magnitude of the force acting on the substrate in dependence on the change in the electrical property.

Where the or each sensor cell comprises a plurality of variable resistors, the variable resistors may be connected in series or in parallel.

The electrical property may comprise one or more of voltage, current, or resistance. The skilled person will be aware of various arraignments of electronic components which may used to measure the electrical property, such as a voltmeter arranged in a voltage divider circuit, for example. The term “voltmeter” used herein should be understood as any arrangement of components that measure voltage.

Where the or each sensor cell comprises a plurality of variable resistors connected in parallel, the system may comprise a reference resistor of fixed resistance for each variable resistor, wherein each reference resistor is connected in series with one of the variable resistors. The one or more measuring devices may comprise a voltmeter for each reference resistor. Each voltmeter may be arranged to measure voltage across one of the reference resistors. The electrical property may be voltage across each reference resistor.

Alternatively, the variable resistors may be connected in parallel and the one or more measuring devices may comprise a voltmeter for each variable resistor, wherein each voltmeter may be arranged to measure voltage across one of the variable resistors. The electrical property may be voltage across each reference resistor.

In other embodiments, the variable resistors may be connected in parallel and the one or more measuring devices may comprise an ammeter for each variable resistor, wherein each ammeter may be arranged to measure current through one of the variable resistors. The electrical property may be current through each reference resistor. The term “ammeter” should be understood any arrangement of components that meaure electrical current.

Where the system comprises the sensor array of the second aspect of the invention, the processor may be configured to determine the distribution of forces on a surface on which the sensor cells of the array are arranged.

A fourth aspect of the invention provides use of the system of the third aspect of the invention to measure force acting on a plantar surface. The use may be to measure shear force and pressure acting on the plantar surface. The sensor cell is intented to be used in a smart insole system to monitor plantar loads of patients with imbalances. The centre of pressure will be calculated from the array of sensor cells to assess the balance of the body and give feedback to a healthcare expert about the progression of care treatment. In one example, the system may be used to measure shear forces on the plantar surface of a sprinter with the aim of modifying the technique of the spinter to minimise lateral shear forces. Lateral shear forces may be undesirable, since they may reduce the efficiency with which a sprinter accelerates. It will be appreciated that the first, second and third aspects of the invention are not limited to any specific use. Other uses of the first, second and/or third aspects of the invention may include: wearable technology; smart insoles; data collection devices for sports; prosthetics; artificial pressure-sensitive skins; surgical robot grasping control; load cells of tri-axial mechanical test machines; industrial robot haptics; and gaming controllers.

The system of the third aspect may, for example, be integrated into an insole of a shoe, or otherwise disposed in contact with a subject's foot.

A fifth aspect of the invention provides a method of manufacturing a sensor cell, the sensor cell comprising:
a conductive elastic substrate;
a plurality of variable resistors each comprising:
   a first electrode;
   a second electrode, wherein the first electrode of each variable resistor is separate and the resistors share a common second electrode; and
   a portion of the conductive elastic substrate, wherein the portion of the substrate is arranged between the electrodes;
   an insulation layer, wherein the second electrode is arranged between the insulation layer and the substrate; wherein
a shear force acting on the substrate causes a distance between the electrodes of a first subset of the resistors to increase and a distance between the electrodes of a second subset of the resistors to decrease, and a pressure acting on the substrate causes a distance between the electrodes of both subsets of resistors to decrease; the method comprising:
forming the first electrodes on the insulation layer using an etching process;
adhering the substrate to the first electrodes; and
adhering the second electrode to the substrate.

The sensor cell of the fifth aspect of the invention may be the sensor cell of the first aspect of the invention (including any optional features thereof).

The method may comprise forming at least part of the substrate using an additive manufacturing process. The additive manufacturing process may be fused filament fabrication (FFF). The method may comprise adjusting a thickness of the substrate and/or adjusting a percentage printing infill of the substrate in dependence on a required sensitivity of the sensor. Varying the thickness of the sensor and/or the percentage infill may adjust the sensitivity of the device. Increasing a thickness of the substrate may improve the suitability of the sensor for higher forces and decreasing a thickness of the substrate may improve the suitability of the sensor for lower forces. Increasing the percentage printing infill may improve the suitability of the sensor for lower forces and decreasing the percentage printing infill may improve the suitability of the sensor for higher forces.

The substrate and the spacer may be formed in a single additive manufacturing session. For example a dual-extrusion method may be used to extrude two different materials (one conducting, and one non-conducting) in a single additive manufacturing session (e.g. FFF). The method may comprise forming a packaging frame for the sensor cell that is co-formed with the spacer. For example, the packaging frame and spacer may be formed from a non-conducting thermoplastic during the same additive manufacturing session that the substrate is formed in.

The sensor cell may comprise a spacer arranged between the substrate and the second electrode, wherein the spacer comprises an open area to allow contact between the substrate and the second electrode. The method may comprise forming the spacer by:
pouring uncured material onto a flat surface;
agitating the uncured material;
curing the material through heating; and
cutting the cured material to form the open area.

The material used to form the spacer may comprise polydimethylsiloxane.

The sensor cell may comprise a bump layer comprising one or more bumps, wherein the method comprises forming the bump layer using a moulding process and coupling the bump layer to the substrate such that each bump exerts a force on the substrate when a corresponding force is exerted on the bump.

The features of each aspect or embodiment may be combined with the features of any other aspect or embodiment.

### BRIEF DESCRIPTION OF THE DRAWINGS

Embodiments of the invention will now be described, by way of example only, with reference to the accompanying drawings:

FIG. 1a shows an isometric exploded view of a sensor cell according to a first example embodiment of the invention;

FIG. 1b shows a further isometric exploded view of the sensor cell;

FIG. 1c shows an exploded side view of the sensor cell;

FIG. 2a shows an isometric view of a first part of a substrate of the sensor cell of FIGS. 1a-c;

FIG. 2b shows a side view of the first part;

FIG. 2c shows a microscopic plan view of the substrate where the percentage infill of the substrate is 100%;

FIG. 2d shows a microscopic plan view of the substrate where the percentage infill of the substrate is 90%;

FIG. 2e shows a microscopic plan view of the substrate where the percentage infill of the substrate is 80%;

FIG. 2f shows a microscopic plan view of the substrate where the percentage infill of the substrate is 70%;

FIG. 2g shows an isometric view of the assembled substrate;

FIG. 2h shows a side view of the assembled substrate;

FIG. 2i shows a further side view of the assembled substrate;

FIG. 3a shows an isometric view of the assembled sensor cell of FIGS. 1a-c;

FIG. 3b shows a further isometric view of the assembled sensor cell;

FIG. 3c shows a plan view of the assembled sensor cell;

FIG. 3d shows a side view of the assembled sensor cell;

FIG. 3e shows a further side view of the assembled sensor cell;

FIG. 3f shows a plan view of the sensor cell next to a coin for scale;

FIG. 4a shows a schematic side view of the sensor cell of FIGS. 1a-c when the sensor cell is at rest;

FIG. 4b shows a further schematic side view of the sensor cell when the sensor cell is at rest;

FIG. 4c shows a schematic side view of the sensor cell with an evenly distributed normal force acting on the sensor cell;

FIG. 4d shows a further schematic side view of the sensor cell with the evenly distributed normal force acting on the sensor cell;

FIG. 4e shows a schematic side view of the sensor cell, showing further compression of the substrate of the sensor cell with the evenly distributed normal force acting on the sensor cell;

FIG. 4f shows a further schematic side view of the sensor cell, showing further compression of the substrate of the sensor cell with the evenly distributed normal force acting on the sensor cell;

FIG. 4g shows a schematic side view of the sensor cell with a shear force in the positive y-direction acting on the sensor cell;

FIG. 4h shows a schematic side view of the sensor cell with a shear force in the negative y-direction acting on the sensor cell;

FIG. 4i shows a schematic side view of the sensor cell with a shear force in the positive x-direction acting on the sensor cell;

FIG. 4j shows a schematic side view of the sensor cell with a shear force in the negative x-direction acting on the sensor cell;

FIG. 5a shows a schematic isometric view of the substrate of the sensor cell of FIGS. 1a-c under the influence of a normal force in the absence of any shear force;

FIG. 5b shows a schematic side view of the substrate of the sensor cell of FIGS. 1a-c under the influence of a normal force in the absence of any shear force;

FIG. 5c shows a further schematic side view of the substrate of the sensor cell of FIGS. 1a-c under the influence of a normal force in the absence of any shear force;

FIG. 5d shows the substrate under the influence of a shear force in the positive y-direction;

FIG. 5e shows the substrate under the influence of a shear force in the negative y-direction;

FIG. 5f shows the substrate under the influence of a shear force in the negative x-direction;

FIG. 5g shows the substrate under the influence of a shear force in the positive x-direction;

FIG. 5h shows an isometric view of the substrate under the influence of a shear force in a direction at 45 degrees to the negative x-direction and negative y-direction;

FIG. 5i shows a side view of the substrate under the influence of a shear force in a direction at 45 degrees to the negative x-direction and negative y-direction;

FIG. 5j shows an isometric view of the substrate under the influence of a shear force in a direction at 45 degrees to the positive x-direction and positive y-direction;

FIG. 5k shows a side view of the substrate under the influence of a shear force in a direction at 45 degrees to the positive x-direction and positive y-direction;

FIG. 6a shows a schematic diagram of a system for measuring force according to an embodiment of the invention;

FIGS. 6b-i each show a plot of a two-dimensional Gaussian function indicating a position and magnitude of a relatively concentrated normal load acting on the sensor cell of FIGS. 1a-c;

FIG. 7 shows an example use of a system of an embodiment of the invention;

FIGS. 8a and 8b illustrate an example method of manufacturing the sensor cell of FIGS. 1a-c;

FIG. 9a shows an apparatus used to validate the performance of the sensor cell of FIGS. 1a-c in measuring normal force;

FIG. 9b illustrates a first configuration of the apparatus;

FIG. 9c illustrates a second configuration of the apparatus;

FIG. 9d shows a set-up of the sensor cell in an example validation study;

FIG. 9e shows a graph of raw data from a cyclic loading test of the validation study;

FIG. 9f shows a graph of raw data from a creep test of the validation study;

FIG. 9g shows a graph of raw data from a stepwise load test of the validation study where the load was applied across the entire surface of the bump layer of the sensor cell;

FIG. 9h shows a graph of raw data from the stepwise load test of the validation study where the load was applied to each bump of sensor cell individually;

FIG. 10a shows an apparatus used to validate the accuracy of an angle of a force vector determined using a simulation of forces acting on the sensor cell of FIGS. 1a-c;

FIG. 10b shows a plot of the magnitude of a force vector applied to the sensor cell at a first angle as calculated using the simulation against the force vector applied at the angle as calculated using the apparatus;

FIG. 10c shows a plot of the magnitude of a force vector applied to the sensor cell at a different angle as calculated using the simulation against the force vector applied at the angle as calculated using the apparatus;

FIG. 11a shows an apparatus used to validate the performance of the sensor cell of FIGS. 1a-c in measuring shear force;

FIG. 11b illustrates the application of varying shear forces to the bump layer of the sensor cell in positive and negative y-directions;

FIG. 11c illustrates the application of varying shear forces to the bump layer of the sensor cell in positive and negative x-directions;

FIG. 11d shows a graph showing the effect of shear force along the x-axis of the sensor cell;

FIG. 11e shows a graph showing the effect of shear force along the y-axis of the sensor cell;

FIG. 12a shows another apparatus used to validate the performance of the sensor cell of FIGS. 1a-c in measuring shear force, with shear force applied by the apparatus to the sensor cell in a first direction;

FIG. 12b shows the apparatus of FIG. 12a, with shear force applied by the apparatus to the sensor cell in a first direction;

FIG. 12c shows raw data from the strain gauge arrangement of the apparatus of FIG. 12a when shear force is applied to the sensor cell along the x-axis by the apparatus of FIG. 12a;

FIG. 12d shows raw data from voltmeters arranged to measure voltage across reference resistors in series with the variable resistors of the sensor cell when shear force is applied to the sensor cell along the x-axis by the apparatus of FIG. 12a;

FIG. 12e shows shear force along the x-axis of the sensor cell as calculated using the apparatus of FIG. 12a and as calculated using the sensor cell following calibration;

FIG. 12f shows raw data from the strain gauge arrangement of the apparatus of FIG. 12a when shear force is applied to the sensor cell along the y-axis by the apparatus of FIG. 12a;

FIG. 12g shows raw data from voltmeters arranged to measure voltage across reference resistors in series with the variable resistors of the sensor cell when shear force is applied to the sensor cell along the y-axis by the apparatus of FIG. 12a;

FIG. 12h shows shear force along the y-axis of the sensor cell as calculated using the apparatus of FIG. 12a and as calculated using the sensor cell following calibration;

FIG. 13 shows a graph showing raw data from a voltmeter arranged to measure voltage across a reference resistor in series with the variable resistors of the sensor cell of FIGS. 1a-c at each step of a stepwise load for a 0.3 mm sensor cell, a 0.4 mm sensor cell, and a 0.7 mm sensor cell;

FIG. 14 shows measured raw data from a voltmeter arranged to measure voltage across a reference resistor in series with the variable resistors of the sensor cell of FIGS. 1a-c at each step of a stepwise load for a sensor cell comprising a substrate with 80% infill and for a sensor cell comprising a substrate with 100% infill;

FIG. 15 shows a sensor cell according to a second example embodiment;

FIG. 16 shows a simulation of the deformation of the sensor cell of FIG. 15 to a shear load;

FIGS. 17a and 17b show a sensor cell according to a third example embodiment;

FIG. 18 shows a simulation of the deformation of the sensor cell of FIGS. 17a and 17b under a shear load;

FIGS. 19a and 19b show a sensor cell according to a fourth example embodiment;

FIG. 20 shows a simulation of the deformation of the sensor cell of FIG. 19 under a shear load;

FIGS. 21, 22 and 23 respectively show results obtained from the sensor cell according to the fourth example embodiment;

FIGS. 24 and 25 show an insole sensor according to an example embodiment comprising sensor cells;

FIG. 26 shows an insole comprising sensor cells and an embedded power source and microcontroller; and

FIG. 27 illustrates a method of making a sensor cell using dual extrusion additive manufacturing.

### DETAILED DESCRIPTION

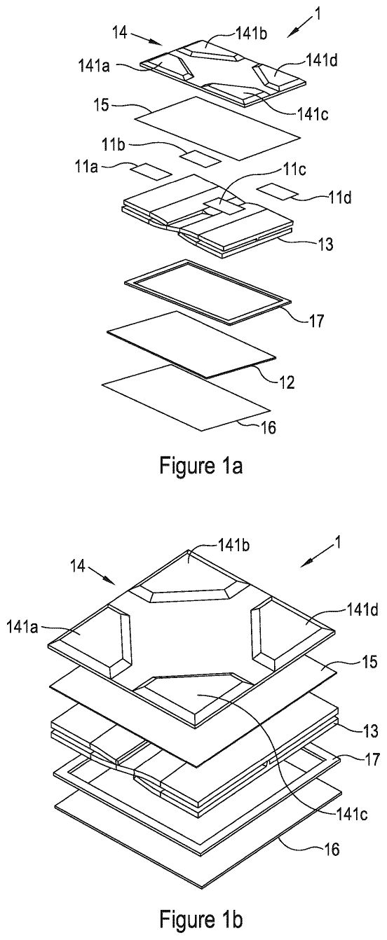

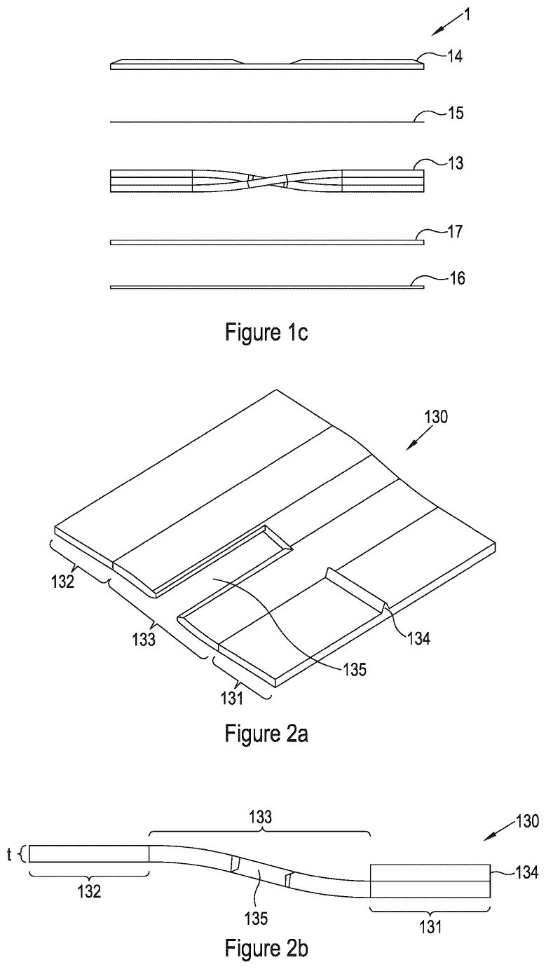

FIG. 1a shows an isometric exploded view of a sensor cell 1 according to a first example embodiment of the invention. The sensor cell 1 comprises four upper electrodes 11a, 11b, 11c, 11d, a single lower electrode 12, and a conductive elastic substrate 13 arranged between the upper and lower electrodes 11, 12. Each upper electrode 11 in combination with the lower electrode 12 and a portion of the substrate 13 provides a variable resistor. In other embodiments, each variable resistor may comprise a separate lower electrode instead of sharing the common lower electrode 12. The sensor cell 1 further comprises a bump layer 14, an upper insulation layer 15, a lower insulation layer 16, and a spacer 17. The upper insulation layer 15 is arranged between the upper electrodes 11 and the bump layer 14, the lower electrode 12 is arranged between the lower insulation layer 16 and the spacer 17, and the spacer 17 is arranged between the lower electrode 12 and the substrate 13. In other embodiments, one or more of the bump layer 14, insulation layers 15, 16 or spacer 17 may not be present.

The terms ‘upper’ and ‘lower’ are used herein to describe components of the sensor cell 1 as they appear in the drawings; it will be appreciated that the terms ‘upper’ and ‘lower’ are not intended to limit the sensor cell 1 to use in a particular orientation. The sensor cell 1 may be used in any suitable orientation, such as sideways or upside down with respect to the orientation of the sensor cell 1 as shown in the drawings.

The bump layer 14 is coupled to the substrate 13, via the upper insulation layer 15 and the upper electrodes 11, such that the bump layer 14 exerts a force on the substrate 13 when a corresponding force is exerted on the bump layer 14. The bump layer 14 comprises four positive bumps 141a, 141b, 141c, 141d with a resulting negative bump between the positive bumps 141.

The spacer 17 extends around the perimeter of the substrate 13 and comprises an open area which allows contact between the substrate 13 and the lower electrode 12 when a force is acting on the substrate 13. When the substrate 13 is at rest, i.e., when there a no external forces acting on the substrate 13, the substrate 13 and the lower electrode 12 are separated by the spacer 17. In this embodiment, the spacer 17 is made from polydimethylsiloxane. In other embodiments, the spacer 17 may be made from an alternative non-conducting material. In some embodiments, the spacer 17 may be made from a non-conducting thermoplastic polyurethane that is additive manufactured (e.g. by fused filament fabrication).

FIG. 1b shows a further isometric exploded view of the sensor cell 1 with the upper and lower electrodes 11, 12 omitted. FIG. 1c shows an exploded side view of the sensor cell 1 with the upper and lower electrodes 11, 12 omitted.

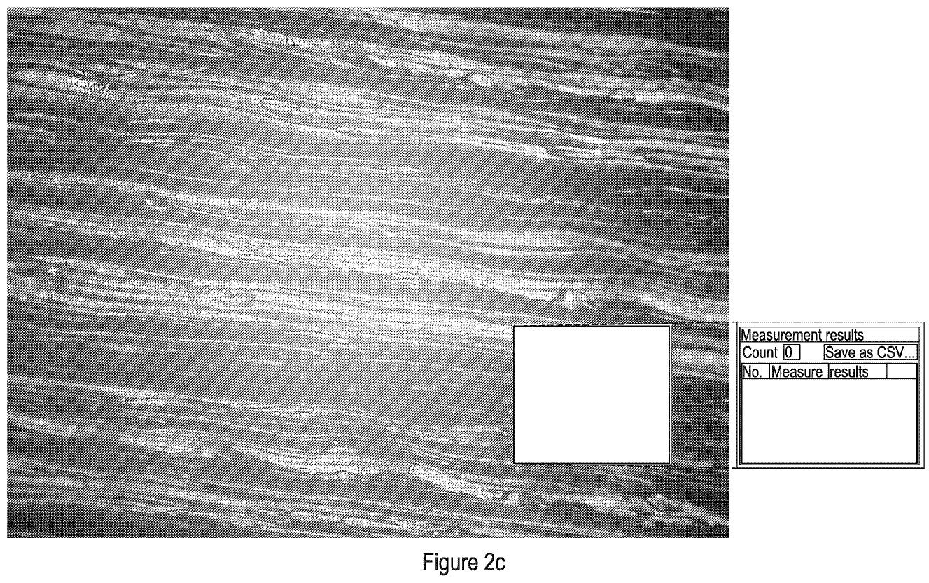

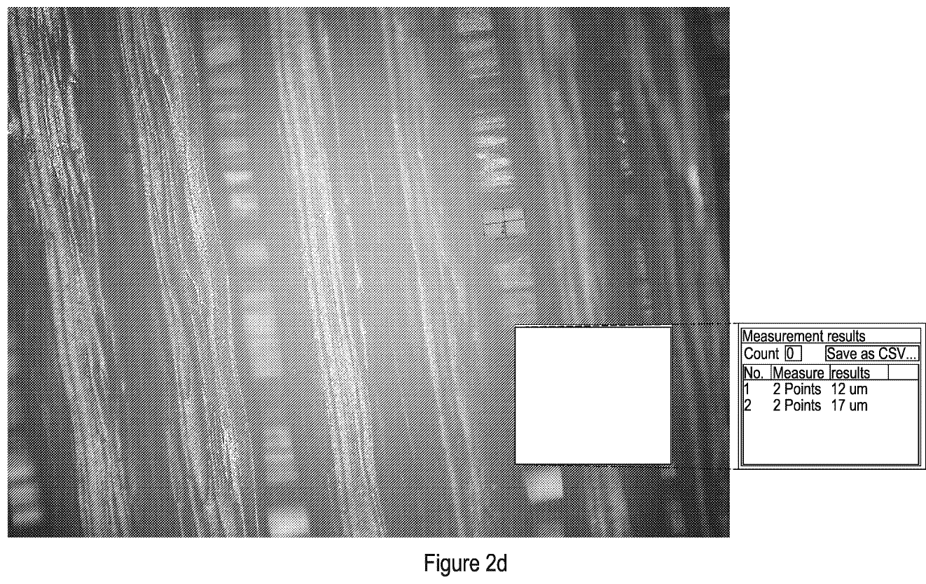

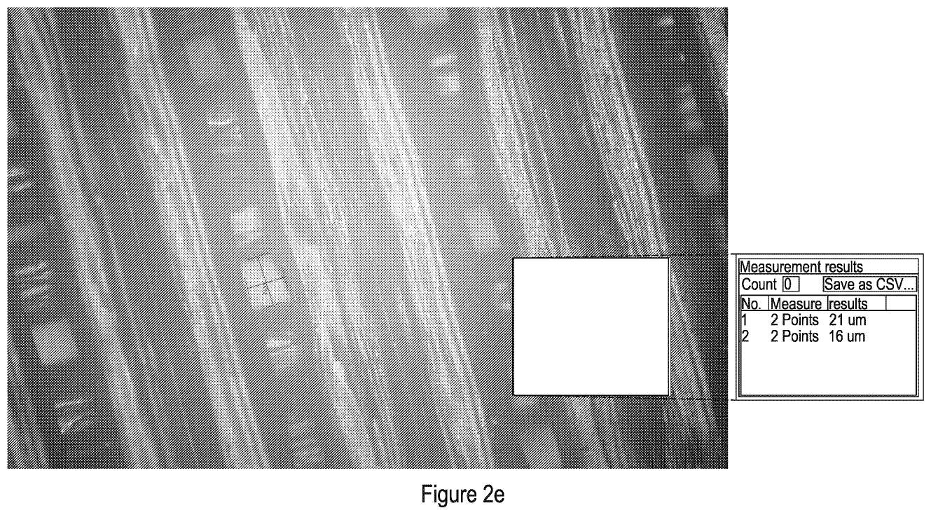

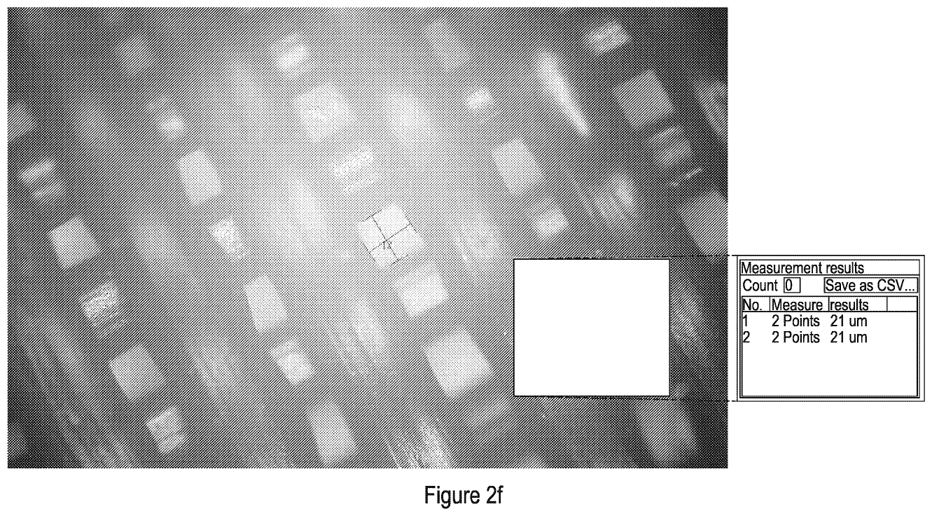

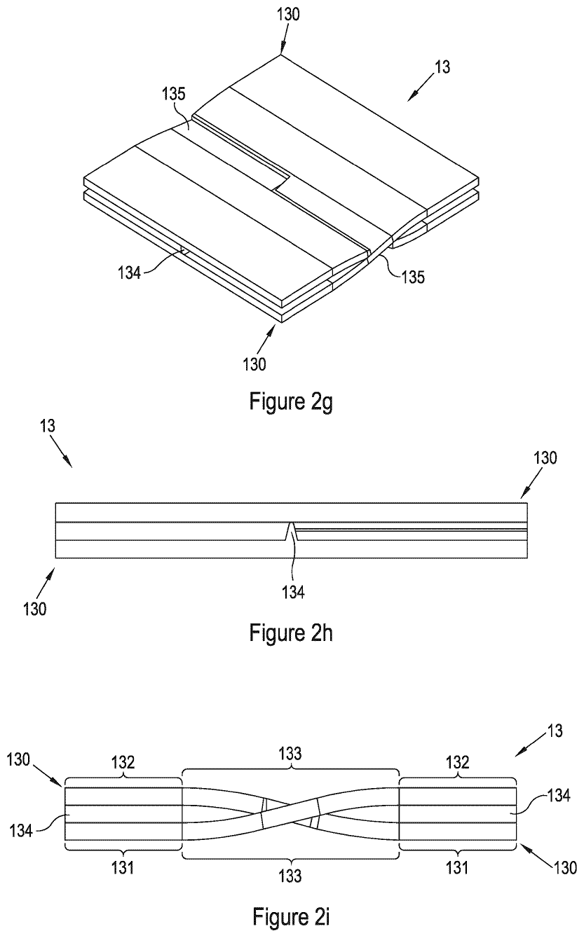

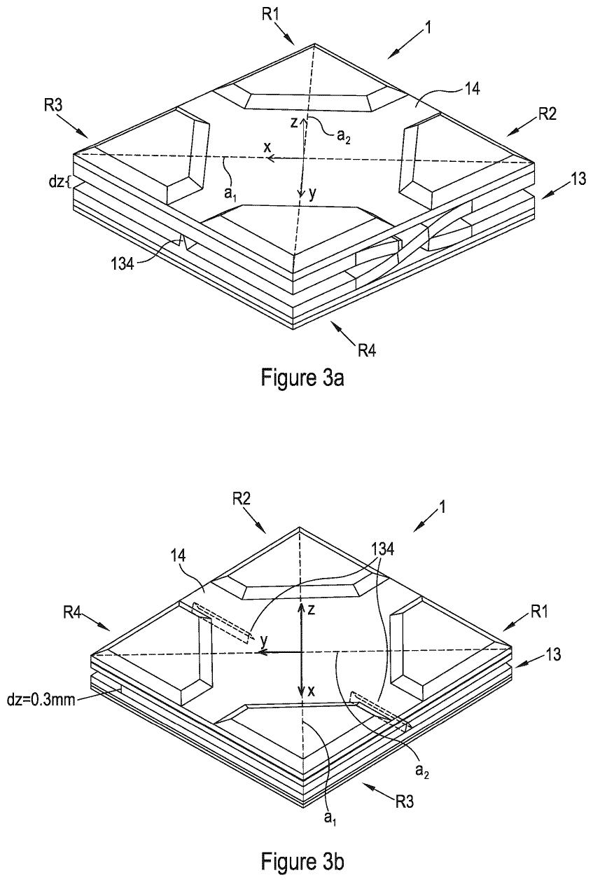

FIG. 2a shows an isometric view of a first part 130 of the substrate 13. FIG. 2b shows a side view of the first part 130. The substrate comprises the first part 130 and an identical second part 130. The first/second part 130 comprises a lower portion 131, an upper portion 132, an inclined portion 133 extending from the lower portion 131 to the upper portion 132, a pivot 134 arranged on the lower portion 131 and extending towards the upper portion 132, and a slot 135 extending inwards from an edge of the inclined portion 133 and extending perpendicularly to the pivot 134. The first and second parts 130 are joined together at the respective slots 135. The design of the substrate 13 takes inspiration from the art of kirigami; the Japanese art of cutting and folding paper.

In this first example embodiment, the first and second parts 130 of the substrate 13 are additive manufactured through fused filament fabrication (FFF) and are made from fibres of thermoplastic polyurethane with a carbon black coating or loaded with carbon black particles. In embodiments where the spacer and the substrate are formed using fused filament fabrication, a dual extrusion method may be used to enable both conducting and non-conducting materials to be printed in a single session. Manufacturing both the substrate and the spacer as a single part in this way makes packaging of the device very simple. Furthermore, correct alignment between the different portions of the device is simplified.

Each part 130 may comprises a single layer of fibres with a 100% infill. In other embodiments, a layer of each part 130 may comprise multiple sub-layers of fibres. Each layer or sub-layer may have a different percentage infill. The percentage infill may be defined with reference to the pitch between adjacent fibres and the fibre diameter. When the fibres are sufficiently close together, they will form a solid layer without holes when the material solidifies. A value of infill of 100% may be defined as a pitch that is equal to the nominal diameter of the fibre. The fibres may sag during printing, with the result that infill values of less than 100% may still result in a solid layer. The distance between adjacent fibres does not depend on fibre orientation. In some embodiments, a 100% infill has 0-5 micron gap distance, 90% 5-15 micron, 80% 15-25 micron, 70% 25-35 micron (measured with digital microscope, KEYENCE, VH-Z100R).

The fibres in example embodiments are orientated at 45 degrees to the edges of the respective part 130, but other fibre orientations can be used. In other embodiments, each part 130 may comprise multiple layers of fibres so as to provide a part of greater thickness, and/or an extruder with a different diameter can be used to print the device. The thickness of the first and second parts 130 is indicated as ‘t’ in FIG. 2b. In other embodiments, the infill of the parts 130 may be different, for example less than 70%, or greater than 70% and less than 100%. In other embodiments, the substrate 13 may be manufactured using any other suitable technique and may be made from a different conductive elastic material.

FIGS. 2c-f each show a microscopic plan view of the substrate 13 of an embodiment of the invention. In each of FIGS. 2c-f, the substrate 13 comprises a single layer comprising two sub-layers of fibres. The fibres in each sub-layer of fibres extend parallel to each other. The fibres of one of the sub-layers of fibres extend perpendicularly to the fibres of the other sub-layer of fibres. In FIG. 2c, the percentage infill of fibres is 100%. In FIG. 2d, the percentage infill is 90%. In FIG. 2e, the percentage infill is 80%. In FIG. 2f, the percentage infill is 70%.

FIG. 2g shows an isometric view of the assembled substrate 13, with the first and second parts 130 joined together. During assembly of the substrate 13, the first and second parts 130 are slotted together at the respective slots 135 and may be fastened together using a suitable adhesive. Once assembled, the fibres of the first and second parts 130 are orientated perpendicularly to each other. FIG. 2h shows a side view of the assembled substrate 13. FIG. 2i shows a further side view of the assembled substrate 13. For clarity, not all the features of the substrate 13 are labelled in FIGS. 2g-i.

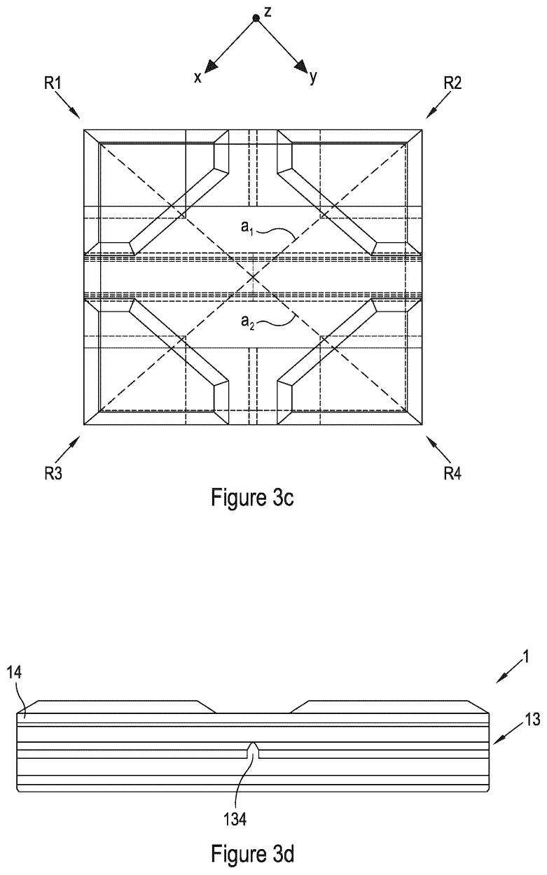

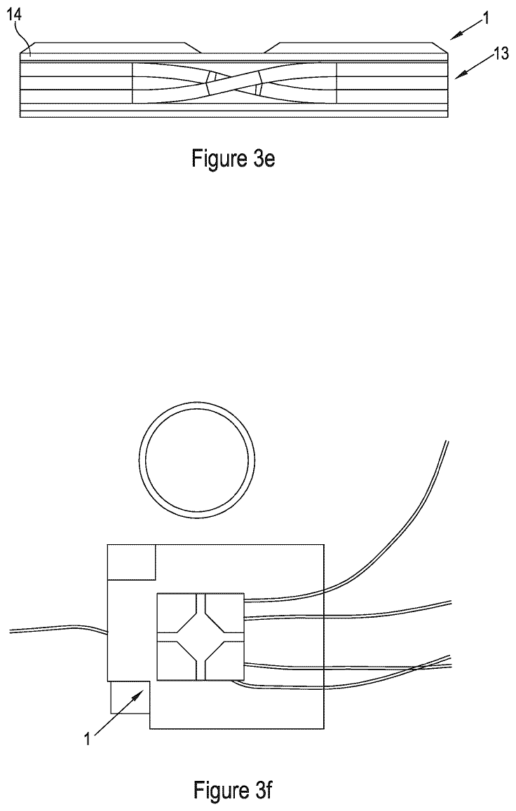

FIG. 3a shows an isometric view of the assembled sensor cell 1. FIG. 3b shows a further isometric view of the assembled sensor cell 1. FIG. 3c shows a plan view of the assembled sensor cell 1. For clarity, not all the features of the sensor cell 1 are labelled in FIGS. 3a-c. The x-, y- and z-axes in FIGS. 3a-c indicate the intented orientation of the sensor cell 1 in use. The configuration of the substrate 13 provides the sensor cell 1 with two pivot axes a₁ and a₂, coincident with the a- and y-xes, respectively. This provides a four-way ‘seesaw’ mechanism.

As described above, the sensor cell 1 comprises four variable resistors, each comprising one of the upper electrodes, a portion of the substrate, and the common lower electrode. The positions of the variable resistors R1, R2, R3, R4, corresponding to the positions of the upper electrodes, are indicated in FIGS. 3a-c. In FIGS. 3a and 3b, the substrate 13 of the sensor cell 1 is at rest, i.e., there are no external forces acting on the substrate 13. In this condition, an air gap ‘dz’ is present between the upper portions of the substrate 13 and the lower portions of the substrate 13. As such, an air gap is present between the electrodes of each variable resistor R1, R2, R3, R4. In this embodiment, as indicated in FIG. 3b, the air gap ‘dz’ when the substrate 13 is at rest is 0.3 mm. In other embodiments, the size of the air gap when the substrate 13 is at rest may be different.

In order to form a conductive path between the electrodes of the variable resistors R1, R2, R3, R4, through the respective portion of the substrate 13, the substrate 13 must deform such that the air gap is closed and contact is made between the upper and lower portions of the substrate 13 at the resistors. The substrate 13 must also deform such that contact is made between the respective lower portion of the substrate 13 and the lower electrode, through the open area of the spacer 17.

A force acting on the substrate 13, via the bump layer 14, may comprise an initial distributed component in the negative z-direction, i.e., an initial distributed normal component, which is evenly distributed across the bump layer 14. For example, where the sensor cell 1 is integrated into an insole of a shoe, or is otherwise disposed in contact with a subject's foot to measure forces on the planar surface, an initial evenly distributed normal force may be exerted on the substrate 13 by virtue of the subject's foot being in the shoe and/or as the subject places their foot in contact with the ground and the user's weight is transferred to the substrate 13 via the bump layer 14. The substrate 13 is configured such the this initial evenly distributed normal force will cause the substrate 13 to compress in the negative z-direction such that the air gap is closed, contact is made between the upper and lower portions of the substrate 13 at each variable resistor R1, R2, R3, R4, and contact is made between the lower portions of the substrate 13 and the common second electrode, thereby forming a conductive path between the upper electrode of each variable resistor R1, R2, R3, R4 and the common second electrode. Further shear force or normal force on the substrate 13 following the initial evenly distributed normal force will cause the substrate 13 to deform in the manner described below. When all forces are removed from the substrate 13, the elastic substrate 13 returns to the rest condition. The spacer 17 improves hysteresis of the sensor cell 1 by returning the cell to an open circuit through separation of the substrate 13 from the lower electrode.

The substrate 13 has a relatively high aspect ratio, with the distance between the upper electrodes is much larger than the thickness of the substrate. The result is that the resistance of the substrate between upper electrodes in the x-y plane is high and resistance in the z-direction (between the upper electrode and underlying ground electrode) is relatively lower (once the air gaps are taken up and the substrate is contacted to the ground electrode). As such, when a conductive path is formed between the electrodes of the resistors, conduction between the electrodes is in the z-direction and any conduction in the x-y plane is negligible.

FIG. 3d shows a side view of the assembled sensor cell 1. FIG. 3e shows a further side view of the assembled sensor cell 1. FIG. 3f shows a plan view of the sensor cell next to a 5 pence coin for scale (which has a diameter of 18 mm). The sensor cell is less than 15 mm across. In other embodiments the sensor cell may be less than 10 mm or less than 5 mm across (e.g. down to 2 mm in size). In some embodiments the sensor cell may be larger than 15 mm across, or larger than 20 mm across. It will be appreciated that these examples are merely illustrative, and the sensor cell 1 may be any suitable size in practice.

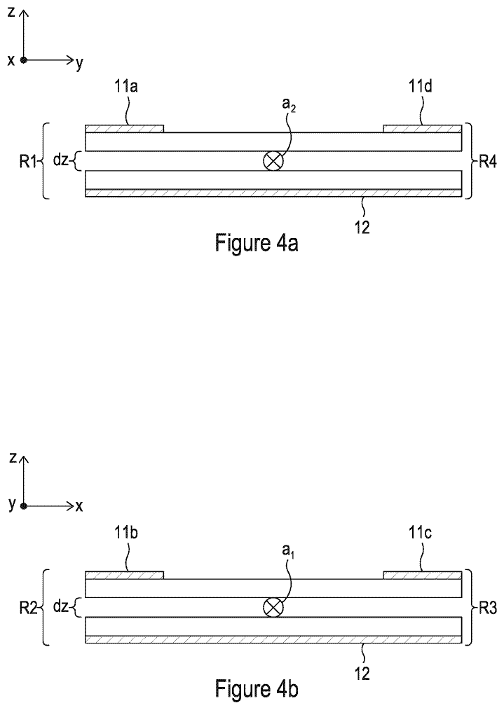

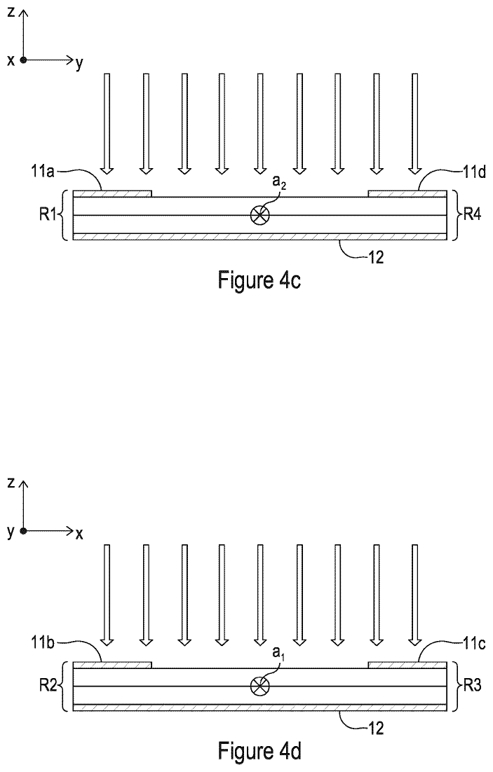

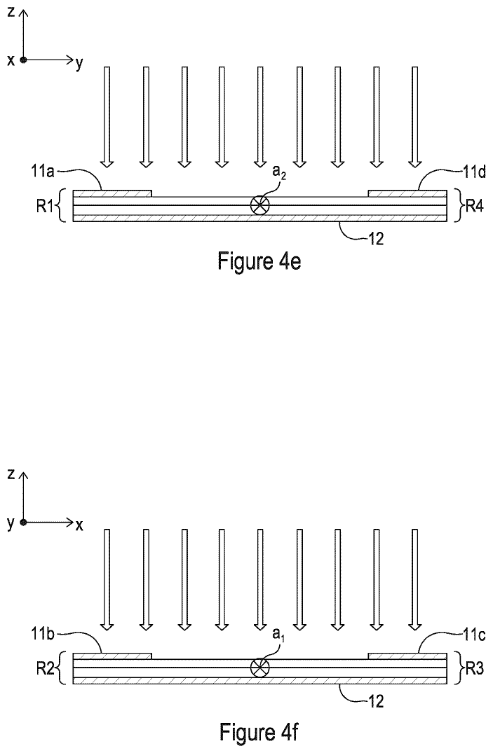

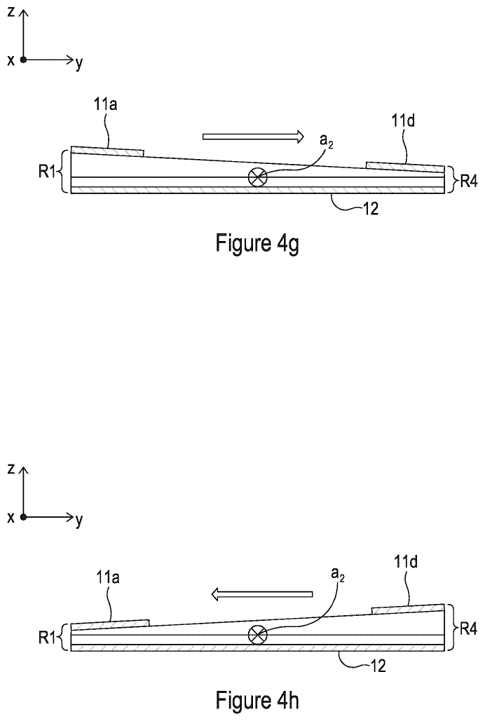

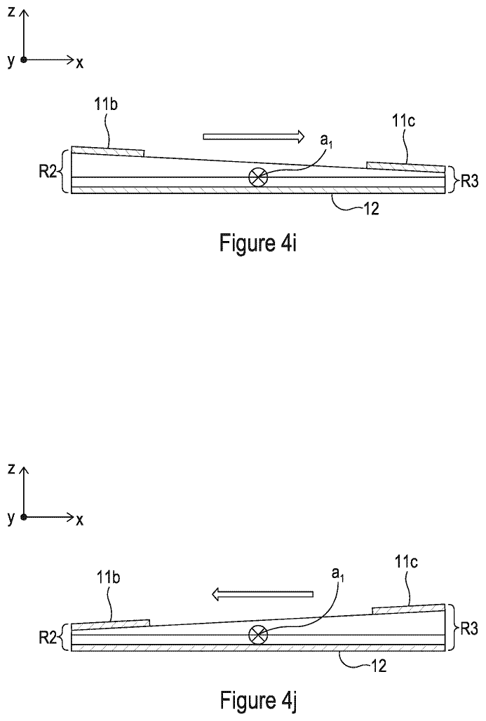

FIGS. 4a-j demonstrate the operation of the sensor cell 1. Each of FIGS. 4a-j show a side view of the sensor cell 1, with the orientation indicated by the axes shown. For clarity, not all parts of the sensor cell 1 are shown and the lower portions 131, upper portions 132 and pivot axes a₁, a₂, of the substrate 13 are represented schematically.

FIGS. 4a and 4b show the sensor cell 1 at rest with the air gap ‘dz’ indicated between the upper portions 132 of the substrate 13 and the lower portions 131 of the substrate 13. As described above with reference to FIGS. 3a-c, a force acting on the substrate 13 in practice may have an initial evenly distributed normal force component. This component is indicated by arrows in FIGS. 4c and 4d. The initial evenly distributed normal force closes the air gap between the upper and lower portions 132, 131 of the substrate, bringing the upper and lower portions 132, 131 and forming a conductive path between the upper and lower electrodes 11, 12 of the variable resistors R1, R2, R3, R4, as shown in FIGS. 4c and 4d. The initial evenly distributed normal force further compresses the substrate 13, causing the distance between the electrodes of each of resistors R1, R2, R3, R4 to decrease, as shown in FIGS. 4e and 4f.

Following the initial evenly distributed normal force, a shear force acting on the substrate 13, via the bump layer, in the positive y-direction will cause the substrate 13 to deform in such a way that a distance between the electrodes of resistor R4 will decrease and a distance between the electrodes of resistor R1 will increase, as shown in FIG. 4g. A shear force acting on the substrate 13 in the negative y-direction will cause the substrate 13 to deform in such a way that a distance between the electrodes of resistor R1 will decrease and a distance between the electrodes of resistor R4 will increase, as shown in FIG. 4h. A shear force acting on the substrate 13 in the positive x-direction will cause the substrate 13 to deform in such a way that a distance between the electrodes of resistor R3 will decrease and a distance between the electrodes of resistor R2 will increase, as shown in FIG. 4i. A shear force acting on the substrate 13 in the negative x-direction will cause the substrate 13 to deform in such a way that a distance between the electrodes of resistor R2 will decrease and a distance between the electrodes of resistor R3 will increase, as shown in FIG. 4j. Additional normal force acting on the substrate 13 in the negative z-direction, further to the initial evenly distributed normal force, will cause the distance between the electrodes of each of resistors R1, R2, R3, R4 to decrease.

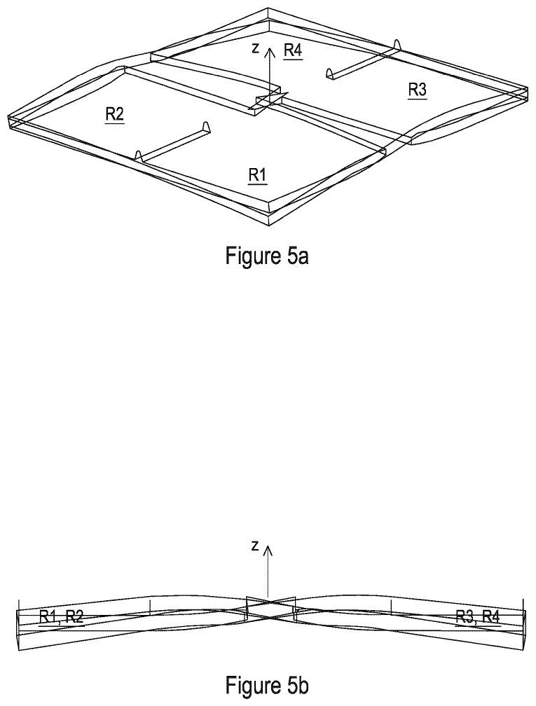

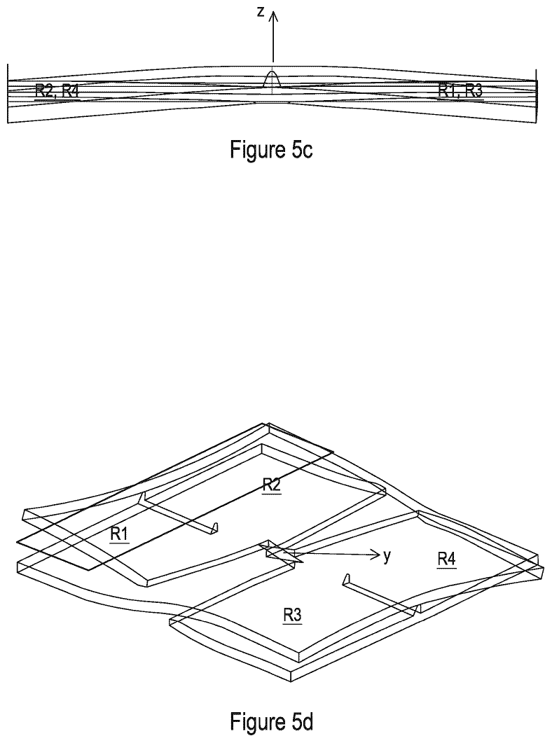

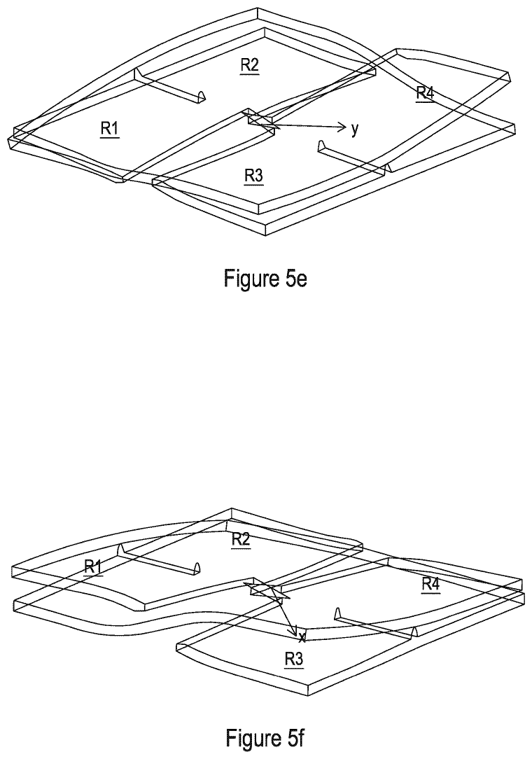

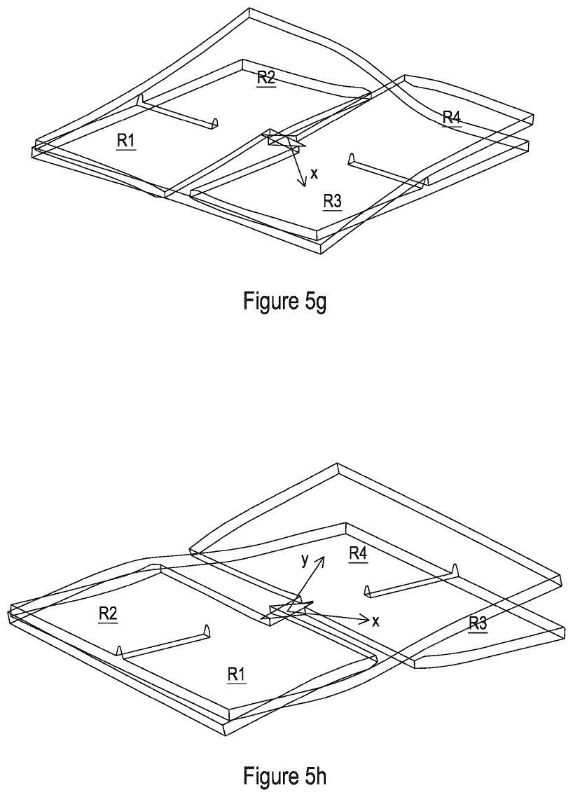

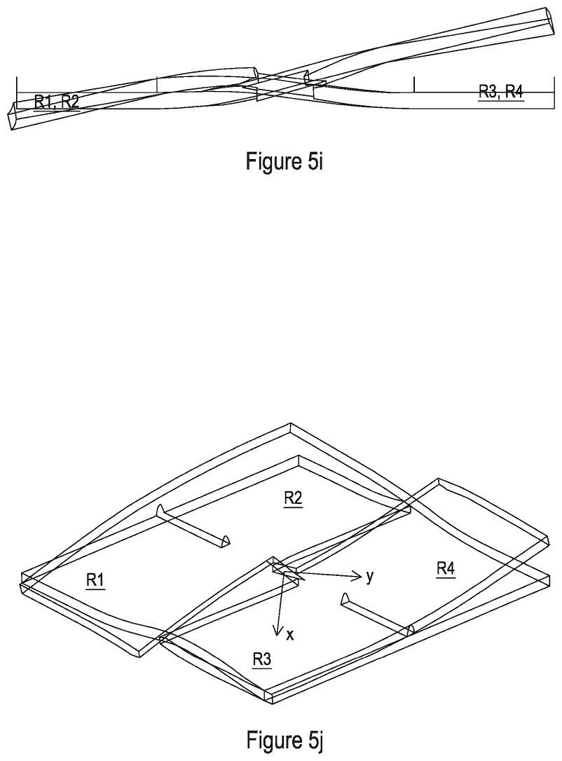

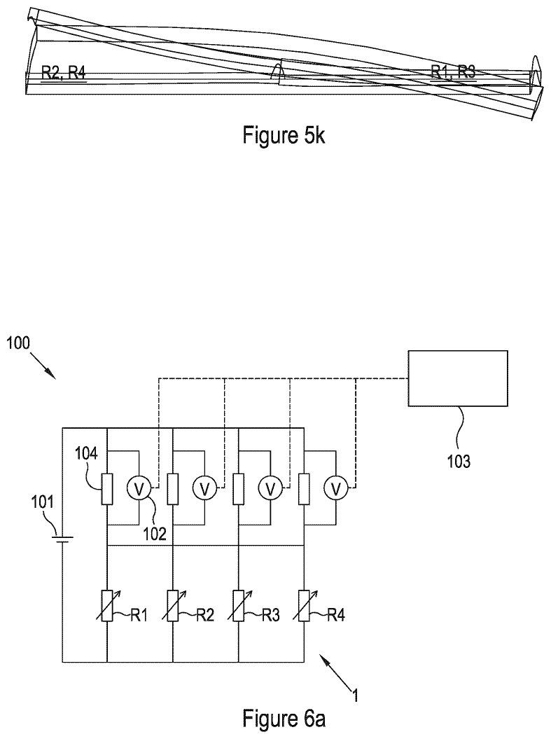

FIGS. 5a-k demonstrate how the substrate 13 deforms when different forces are applied to the substrate 13. For reference, the corresponding positions of the variable resistors R1, R2, R3, R4 and the x-, y- and z-axes are shown where appropriate. FIGS. 5a-k are each generated from a finite element analysis of the substrate 13. FIGS. 5a-k are provided as an indication of how the substrate deforms under load. FIGS. 5a-k should not be interpreted literally; for example, where the figures appear to show separation of the upper and lower portions of the substrate under the influence of a shear force, it will be appreciated that such separation does not occur in practice when there is an initial normal load sufficient to keep the gaps closed. FIGS. 4a-j show how the substrate actually deforms in practice (when a sufficient normal load is present to close the gaps in the device).

FIGS. 5a-c show the substrate under the influence of a normal force in the negative z-direction in the absence of any shear force. FIG. 5d shows the substrate under the influence of a shear force in the positive y-direction. FIG. 5e shows the substrate under the influence of a shear force in the negative y-direction. FIG. 5f shows the substrate under the influence of a shear force in the negative x-direction. FIG. 5g shows the substrate under the influence of a shear force in the positive x-direction. FIG. 5h shows an isometric view of the substrate under the influence of a shear force in a direction at 45 degrees to the negative x-direction and negative y-direction. FIG. 5i shows a side view of the substrate under the influence of a shear force in a direction at 45 degrees to the negative x-direction and negative y-direction. FIG. 5j shows an isometric view of the substrate under the influence of a shear force in a direction at 45 degrees to the positive x-direction and positive y-direction. FIG. 5k shows a side view of the substrate under the influence of a shear force in a direction at 45 degrees to the positive x-direction and positive y-direction.

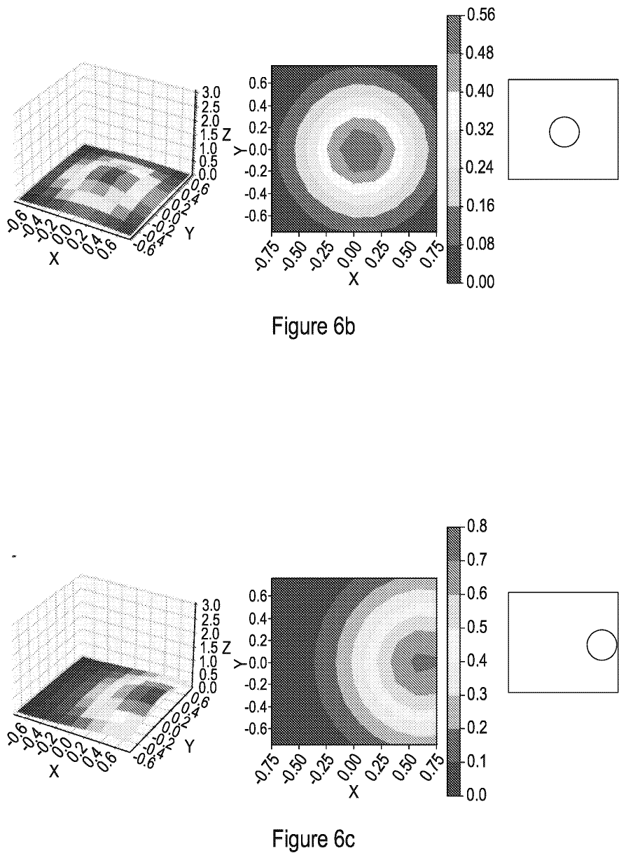

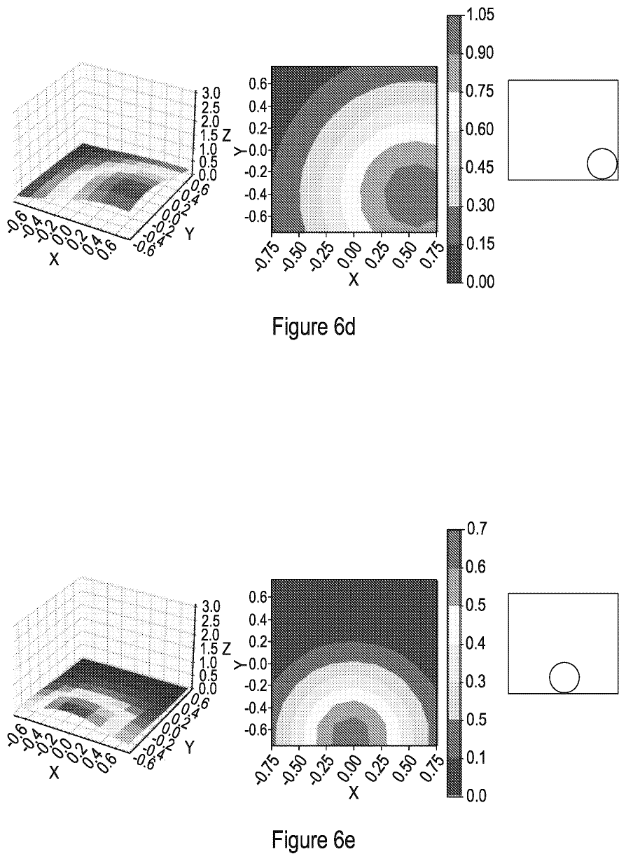

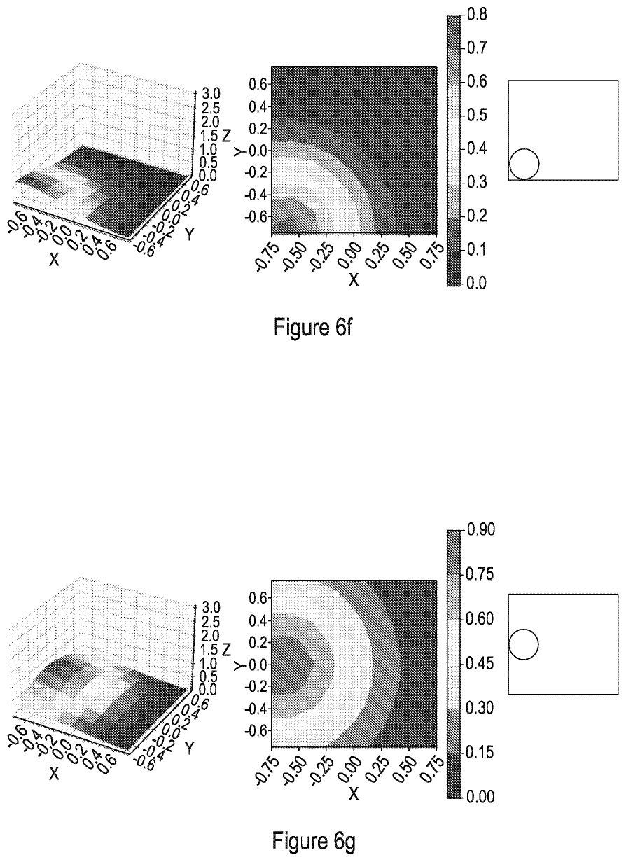

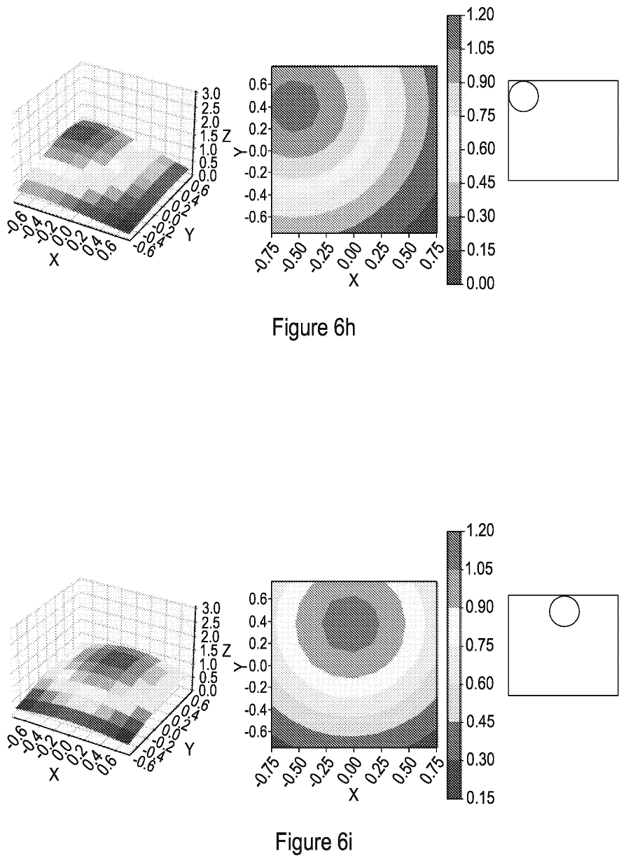

FIG. 6a shows a schematic diagram of an example system 100 for measuring force according to an embodiment of the invention. The system 100 comprises the sensor cell 1 of FIG. 1, a DC power source 101, four voltmeters 102, a processor 103, and four reference resistors 104 of fixed resistance. The sensor cell 1, power source 101, voltmeters 102, and reference resistors 104 are represented schematically in a circuit diagram. The sensor cell 1 is represented by the four variable resistors R1, R2, R3, R4. Each reference resistor 104 is connected in series with one of the variable resistors R1, R2, R3, R4. The low voltage side of the reference resistors may be grounded. The sensor cell 1 may be calibrated to account for environmental effects, such as temperature, on the reference resistors. The processor 103 may be configured to correct measurements to account for temperature variations, for example. Each voltmeter 102 is arranged to measure voltage across one of the reference resistors 104. The processor 103 is configured to determine the magnitude of a force acting on the substrate of the sensor cell 1 in dependence on the change in voltage across each reference resistor 104. This illustration is merely schematic—other arrangements may be use to infer changes in resistance for each of the resistors R1-R4. For example, a full Wheatstone bridge or half Wheatstone bridge may be implemented for each variable resistor.

A decrease in resistance of one of the variable resistors will result in an increase in voltage across the reference resistor connected in series with the variable resistor as measured by the voltmeter arranged to measure the voltage across the reference resistor. As described above, an initial evenly distributed normal force acting on the bump layer 14 will cause a conductive path to be formed through each variable resistor R1, R2, R3, R4.

Referring to FIGS. 4a-j and 6a, a shear force acting on the bump layer 14 in the positive y-direction will cause the substrate 13 to deform in such a way that a distance between the electrodes of resistor R4 will decrease and a distance between the electrodes of resistor R1 will increase. This will cause the resistance of resistor R4 to decrease (see equation (1) above, with L representing the distance between electrodes) and the resistance of resistor R1 to increase, thereby resulting in an increase in voltage measured by the voltmeter arranged to measure voltage across the reference resistor connected in series with resistor R4 and a decrease in voltage measured by the voltmeter arranged to measure voltage across the reference resistor connected in series with resistor R1.

A shear force acting on the bump layer 14 in the negative y-direction will cause the substrate 13 to deform in such a way that a distance between the electrodes of resistor R1 will decrease and a distance between the electrodes of resistor R4 will increase. This will cause the resistance of resistor R1 to decrease (see equation (1) above) and the resistance of resistor R4 to increase, thereby resulting in an increase in voltage measured by the voltmeter arranged to measure voltage across the reference resistor connected in series with resistor R1 and a decrease in voltage measured by the voltmeter arranged to measure voltage across the reference resistor connected in series with resistor R4.

A shear force acting on the bump layer 14 in the positive x-direction will cause the substrate 13 to deform in such a way that a distance between the electrodes of resistor R3 will decrease and a distance between the electrodes of resistor R2 will increase. This will cause the resistance of resistor R3 to decrease (see equation (1) above) and the resistance of resistor R2 to increase, thereby resulting in an increase in voltage measured by the voltmeter arranged to measure voltage across the reference resistor connected in series with resistor R3 and a decrease in voltage measured by the voltmeter arranged to measure voltage across the reference resistor connected in series with resistor R2.

A shear force acting on the bump layer 14 in the negative x-direction will cause the substrate 13 to deform in such a way that a distance between the electrodes of resistor R2 will decrease and a distance between the electrodes of resistor R3 will increase. This will cause the resistance of resistor R2 to decrease (see equation (1) above) and the resistance of resistor R3 to increase, thereby resulting in an increase in voltage measured by the voltmeter arranged to measure voltage across the reference resistor connected in series with resistor R2 and a decrease in voltage measured by the voltmeter arranged to measure voltage across the reference resistor connected in series with resistor R3.

A normal force, following the initial evenly distributed normal force, acting on the bump layer in the negative z-direction will cause the distance between the electrodes of each of resistors R1, R2, R3, R4 to decrease. This will cause the resistance of each of the resistors R1, R2, R3, R4 to decrease, thereby resulting in an increase in voltage measured by the voltmeter arranged to measure voltage across the reference resistor connected in series with each of the resistors R1, R2, R3, R4.

The processor 103 is configured to determine the force(s) acting on the substrate of the sensor cell from the voltages measured by the voltmeters.

$${F}_{x} ∝ {V}_{R 3} - {V}_{R 2}$$

$${F}_{y} ∝ {V}_{R 4} - {V}_{R 1}$$

$${F}_{z} ∝ {V}_{R 1} + {V}_{R 2} + {V}_{R 3} + {V}_{R 4}$$

With V_{Rn} denoting the voltage across the reference resistor in series with variable resistor Rn.

The above equations enable the shear forces in x and y, and the normal force in z, to be determined, once the sensor is calibrated (to determine an appropriate constant of proportionality). The magnitude of the force acting along the y-axis is determined from the difference between the voltages measured by the voltmeter arranged to measure voltage across the reference resistor connected in series with resistor R1 and the voltmeter arranged to measure voltage across the reference resistor connected in series with resistor R4. The magnitude of the force acting along the x-axis is determined from the difference between the voltages measured by the voltmeter arranged to measure voltage across the reference resistor connected in series with resistor R2 and the voltmeter arranged to measure voltage across the reference resistor connected in series with resistor R3. The magnitude of the force acting along the z-axis is determined from the sum of the voltages measured by the voltmeters. In other embodiments, an alternative algorithm may be used to determine the forces acting on the substrate using the voltages measured by the voltmeters.

The processor 103 is configured to determine the components of the total force acting on the substrate along the x-, y- and z-axes and determine the total force vector r, including magnitude and angle to the x-y plane of the substrate, from the three components Fₓ, F_{y}, F_{z}. The spherical coordinates of a point in the ISO convention for physics (radius r, inclination θ, azimuth φ) can be obtained from the x, y and z components of the force:

$$r = \sqrt{{F}_{x}^{2} + {F}_{y}^{2} + {F}_{z}^{2}}$$

$$θ = arccos \frac{{F}_{z}}{r}$$

$$ϕ = \{ \begin{matrix}arctan \frac{{F}_{y}}{{F}_{x}} & if x \geq 0 \\ arctan \frac{{F}_{y}}{{F}_{x}} + π & if x < 0\end{matrix}$$

The sensor signals can be used to visualize forces measured by the sensor. A vector determined from the force components may be visualised for example. A heatmap visualisation may be generated. A heatmap of any resolution can be generated from the sensor signals, according to the following approach:

A 2D mesh grid can be be created, and a 2D Gaussian visualised, which is determined in reliance on the forces measured by the 2D sensor:

$$Gaussian ( x , y , A , σ ) = {Ae}^{- \frac{{x}^{2} + {y}^{2}}{2 {σ}^{2}}}$$

$$A = {F}_{z}$$

The height of the Gaussian may be determined with reference to the force component in the z direction. The width σ of the Guassian could, for example, be determined with reference to the shear components (e.g. a vector sum of x and y components), or may have a predetermined value.

It will be appreciated that the arrangement of the variable resistors R1, R2, R3, R4 connected in parallel and the arrangement of reference resistors and voltmeters is merely an illustrative example of how changes in the resistance of the variable resistors R1, R2, R3, R4 can be used to infer the magnitude and direction of force(s) acting on the substrate.

In some embodiments, the processor may be configured to operate in a different mode (which may be termed “force localisation”, different from with the above “shear sensing” mode), in which the position of a relatively concentrated normal load is determined with reference to the voltages V_{R1}, V_{R2}, V_{R3}, V_{R4}. The processor may be configured to automatically switch between the shear sensing and force localisation modes. The processor may be configured to switch modes in response to an input from a user. The processor may be configured to determine the position of the load with respect to the x-axis of the sensor cell, xₚₒₛ, and with respect to the y-axis of the sensor cell, yₚₒₛ, using the following:

$${x}_{pos} ∝ \frac{{V}_{R 2} - {V}_{R 3}}{{V}_{R 2} + {V}_{R 3}}$$

$${y}_{pos} ∝ \frac{{V}_{R 1} - {V}_{R 4}}{{V}_{R 1} + {V}_{R 4}}$$

The magnitude of the load may be determined from the sum of the voltages V_{R1}, V_{R2}, V_{R3}, V_{R4}:

$${F}_{z} ∝ {V}_{R 1} + {V}_{R 2} + {V}_{R 3} + {V}_{R 4}$$

The x-y position and magnitude of the load may be visulaised using a two-dimensional Gaussian function positioned at xₚₒₛ, yₚₒₛ with the width of the Gaussian scaled with F_{z}. In a validation study, a spherical indentor with a diameter of 4 mm was used to apply an equal normal force to the sensor cell at different x-y positions. FIGS. 6b-i each show a plot of the two-dimensional Gaussian function alongside a plan view of the plot and an indication of the x-y position of the indentor on the sensor cell (the black square representing the x-y plane of the sensor cell and the white circle indicating the position of the indentor).

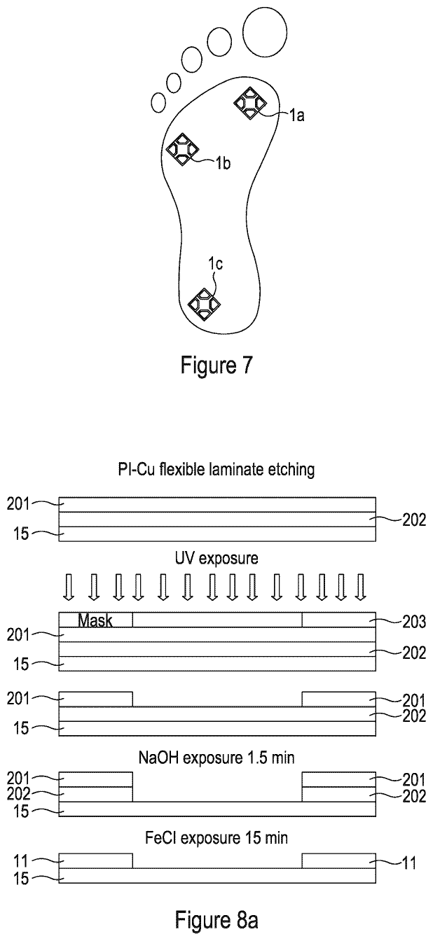

FIG. 7 shows an example use of a system of an embodiment of the invention. In this embodiment, the system comprises all the features of the system 100 of FIG. 6 and additionally comprises further sensor cells. In this example, the system comprises three sensor cells 1a-c. In other examples, more or fewer sensor cells may be provided. The sensor cells 1a-c are arranged proximate to the plantar surface of a human foot, such that the system is arranged to measure force(s) acting on the plantar surface. The sensor cells 1a-c may be imbedded in the sole of shoe or otherwise disposed in contact with the plantar surface. For each sensor cell 1a-c, the system comprises the arrangement of voltmeters 102 and reference resistors 104 as shown in FIG. 6. Each voltmeter 102 of each sensor cell 1a-c is in communication with the processor 103 and the processor 103 is configured to determine the magnitude of a force acting on the substrate of each sensor cell 1a-c in dependence on the change in voltage across each reference resistor 104 of each sensor cell 1a-c as described above. In this way, the processor can determine the distribution of force(s) on the plantar surface, as well as the centre of pressure on the plantar surface and the total moment acting on the plantar surface.

It will be appreciated that the example of FIG. 7 is merely illustrative and that a system of an embodiment of the invention may be used to measure force and/or force distribution in any suitable application.

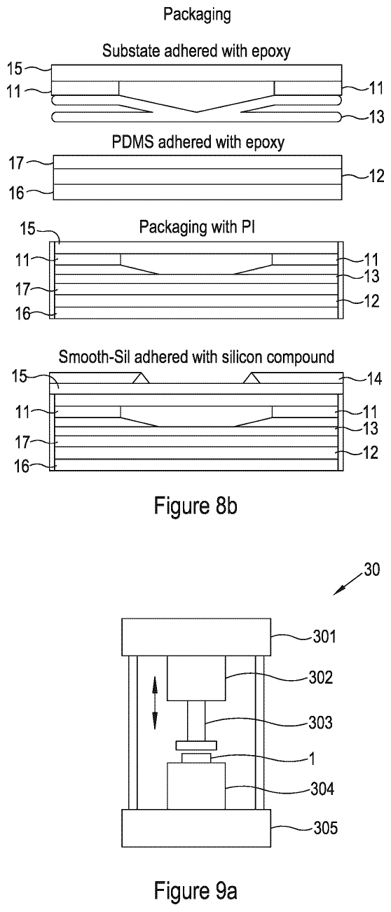

FIGS. 8a and 8b illustrate a method of manufacturing the sensor cell 1 of FIG. 1. The method begins by forming the upper electrodes 11 on the upper insulation layer 15. This first comprises providing a laminate comprising, in order from the lower-most layer upwards, a first layer which will become the upper insulation layer 15 of the complete sensor cell, an electrode layer 202, and a photo sensitive resist film 201. The insulation layer 15 comprises Kapton tape and the electrode layer 202 comprises copper tape. The layers of the laminate are adhered together.

A mask 203 is then positioned on the photo sensitive resist film 201. The mask 203 comprises opaque portions, corresponding to the intended positions of the upper electrodes 11 on the upper insulation layer 15, and transparent portions between the opaque portions. The mask 203 is then exposed to UV radiation.

The laminate is then exposed to a positive developer comprising NaOH for 1.5 minutes to remove all of the resist film 201 except at the intended positions of the upper electrodes 11. The laminate is then exposed to FeCl acid for 15 minutes leaving behind the upper electrodes 11 on the insulation layer 15. A resist stripping step is used to strip the developed resist film 201.

The upper portions of the substrate 13 are then adhered to the upper electrodes 11 with a suitable epoxy (see FIG. 8b). The substrate 13 is formed separately from thermoplastic polyurethane with a carbon black coating/loading using an additive manufacturing process, such as FFF, as described above with reference to FIG. 2a.

A further laminate comprising, in order from the lower-most layer upwards, a first layer which will become the lower insulation layer 16 of the complete sensor cell, the lower electrode 12, and the spacer 17 is then provided. The spacer 17 is produced separately by: pouring uncured polydimethylsiloxane (PDMS) onto a flat surface; agitating the uncured PDMS using an orbital shaker; curing the PDMS through heating in a convection oven at 80° C. for approximately 1 hour 30 minutes; and cutting the cured PDMS to form the open area of the spacer 17 using a square shaped hole puncher. The layers of the further laminate are adhered together using a suitable epoxy.

The substrate 13, with the upper electrodes 11 and the upper insulation layer 15, is then stacked on top of the further laminate. The bump layer 14 is then adhered to the top of the upper insulation layer 15. The bump layer 14 is formed separately from Silicone MoldMax 10T using a suitable moulding process to form the bumps.

It will be appreciated that the method of FIGS. 8a and 8b is merely illustrative and any additional or alternative process(s) and/or material(s) may be used to manufacture the sensor cell.

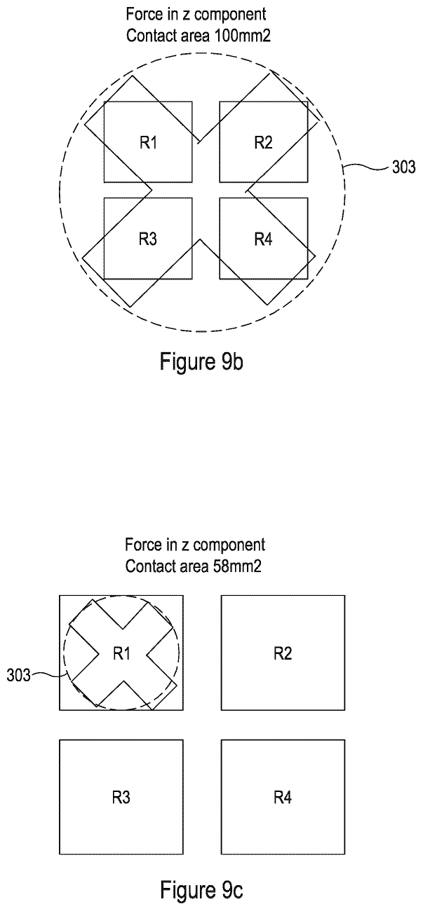

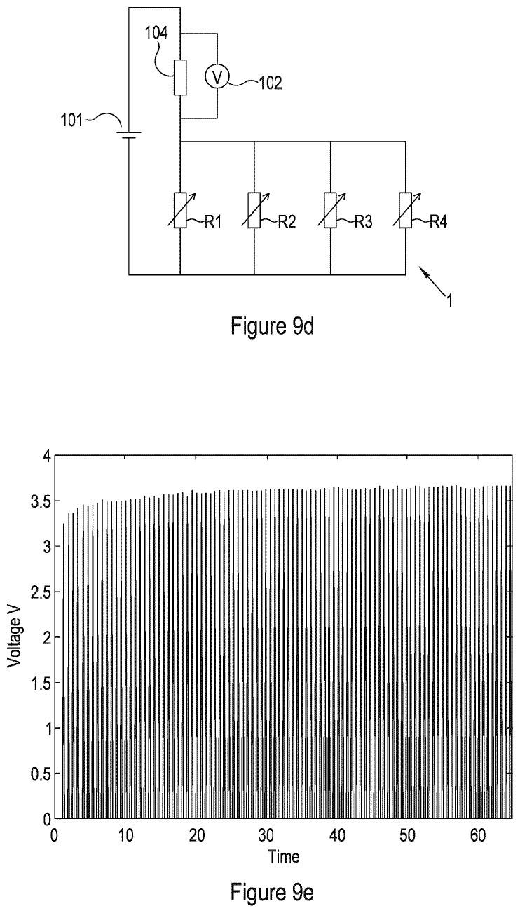

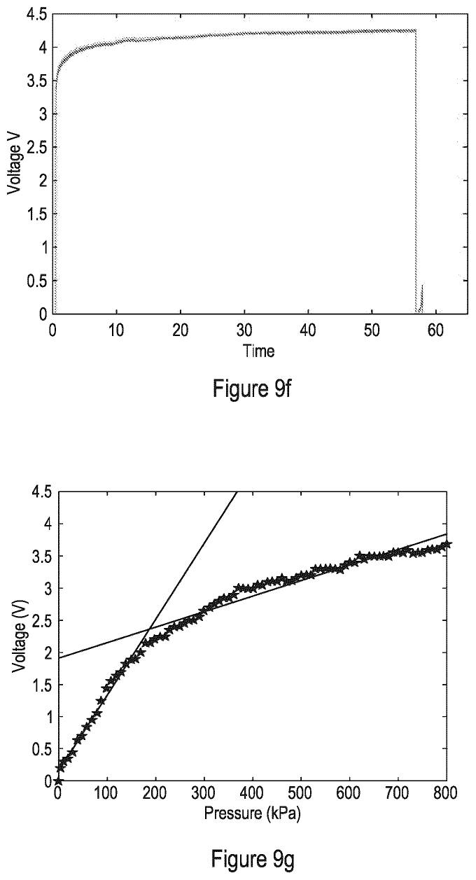

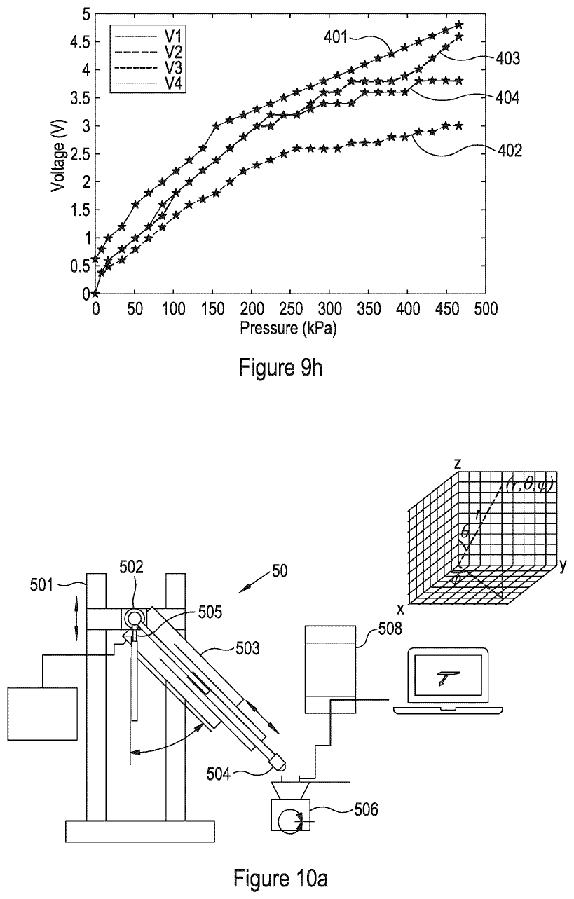

FIG. 9a shows an apparatus 30 used to validate the performance of the sensor cell 1 of FIG. 1 in measuring normal force. The apparatus 30 may also be used to calibrate the sensor cell 1. The apparatus 30 comprises a holder 301, a load cell 302, an indentor 303, a slab 304, and a metal plate 305. In this example, the slab 304 comprises silicone to mimic plantar forces applied to the sole of a shoe. In other examples, where the sensor cell 1 is used in an application other than measuring plantar forces, the slab 304 may comprise a suitable alternative material. The slab 304 is arranged on the metal plate 305 and the sensor cell 1 is arranged on the slab 304. The load cell 302 is arranged to apply a normal force to the bump layer of the sensor cell 1 via the indentor 303.

FIG. 9b illustrates a first configuration of the apparatus 30. In this configuration, the indentor 303 is arranged to apply a normal load distributed across the entire surface of the bump layer of the sensor cell. This causes the distance between the electrodes of each variable resistor R1, R2, R3, R4 of the sensor cell to decrease by the same amount.

FIG. 9c illustrates a second configuration of the apparatus 30. In this configuration, the indentor 303 is arranged to apply a normal load to the bump of the sensor cell arranged above the upper electrode of variable resistor R1. This causes the distance between the electrodes of resistor R1 to decrease while the distance between the electrodes of the other variable resistors remains unchanged. The position of the sensor cell 1 on the slab 304 can be adjusted to align a different bump of the sensor cell with the indentor 303, thereby allowing a normal force to be applied to each individual bump in turn.

In an example validation study, the sensor cell was set up as shown in FIG. 9d. In FIG. 9d, the sensor cell 1 is represented by the four variable resistors R1, R2, R3, R4 arranged in parallel. A reference resister 104 of fixed resistance is arranged in series with the four variable resistors R1, R2, R3, R4. A voltmeter 102 is arranged to measure the voltage across the reference resistor 104. A power source 101 is arranged to provide a voltage across the reference resistor 104 and the variable resistors R1, R2, R3, R4. The skilled person will appreciate that a decrease in resistance of one or more of the variable resistors will result in an increase in voltage across the reference resistor as measured by the voltmeter.

The power source 101 was arranged to supply a 0.3 A current at 5V across the reference resistor 104 and variable resistors R1, R2, R3, R4 of the sensor cell 1. In a first test, a cyclic load of 20N, held for 500 ms during each cycle, for 200 cycles was applied to the bump layer of the sensor cell by the indentor 303. In a second test, creep was investigated by applying a constant load of 20N to the bump layer for 57 mintues. In a third test, the sensitivity of the sensor cell was investigated by applying a stepwise load ranging from IN to 80N in increments of IN. In the first, second and third tests, the loads were applied across the entire surface of the bump layer of the sensor cell using the arrangement of FIG. 9b. The loads were distributed over a contact area of 100 mm². The third test was also carried out using the arrangement of FIG. 9c to apply the stepwise load to each bump individually over a contact area of 58 mm². Zero drift time was also investigated by supplying constant current for 1 hour.

The performance of the sensor cell as characterised by the validation study is shown in Table 1:

TABLE 1

| Performance | Specification | |
|---|---|---|
| Hysteresis | ±0-2% | |
| Response | 100 | ms |
| Recovery | 150 | ms |
| Repeatability | ±4% | |
| Fatigue resistance | 100 | cycles+ |
| Relaxation | ±5%/30 | min |

| | |
|---|---|
| Zero drift time | ±0.5%/hr |

FIG. 9e shows a graph of the raw data from the cyclic loading test of the validation study. The graph shows the voltage measured by the voltmeter 102 at each load cycle.

FIG. 9f shows a graph of the raw data from the creep test of the validation study. The graph shows the voltage measured by the voltmeter 102 throughout the 57 minutes of constant loading.

FIG. 9g shows a graph of the raw data from the stepwise load test of the validation study where the load was applied across the entire surface of the bump layer of the sensor cell. The graph shows voltage measured by the voltmeter 102 at each load step. Voltage increase is linear in two regions, one between approximately 0-200 kPa, with a gradient of approximately 100 Pa/V, and another from approximately 200-800 kPa, with a gradient of approximately 375 Pa/V. This demonstrates that the cell is almost four times more sensitive to low pressures, i.e., less than or equal to approximately 200 kPa, compared to higher pressures, i.e., greater than approximately 200 kPa.

FIG. 9h shows a graph of the raw data from the stepwise load test of the validation study where the load was applied to each bump of sensor cell individually. The graph shows voltage measured by the voltmeter 102 at each load step for the bumps corresponding to: variable resistor R1 (line 401); variable resistor R2 (line 402); variable resistor R3 (line 403); and variable resistor R4 (line 404). Similarly to the results where the load was applied across the entire surface of the bump layer, the results of FIG. 8g show to approximate regions of linearity.

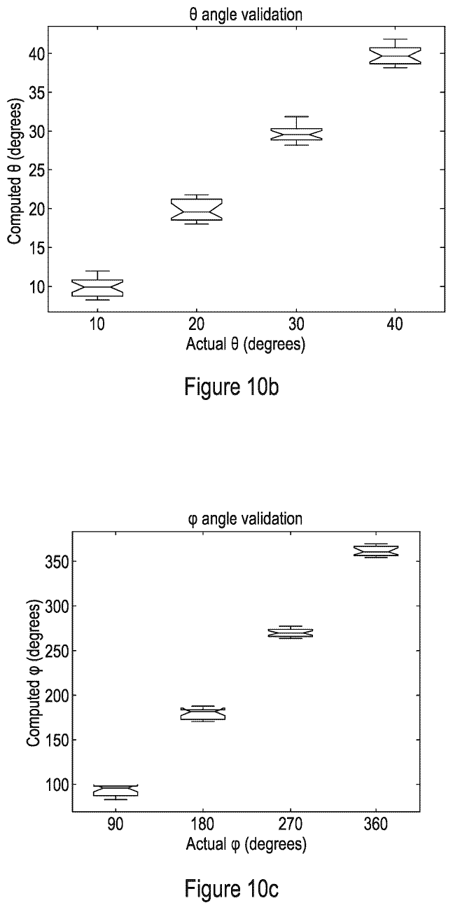

FIG. 10a shows an apparatus 50 used to validate the accuracy of an angle of a force vector determined using a simulation of forces acting on the sensor cell 1 of FIG. 1. The apparatus 50 may also be used to calibrate the sensor cell 1. The apparatus 50 comprises: a frame 501; a hinge joint 502 coupled to the frame 501; a linear actuator 503 coupled to the hinge joint 502; an indentor 504 coupled to the linear actuator 503; a first goniometer 505 configured to measure an angle of the linear actuator 503 with respect to the frame 501; a rotating platform 506; a second goniometer (not shown) configured to measure an angle of rotation of the rotating platform 506; and a computer 508.

In an example validation study, the sensor cell 1 was placed on the rotating platform 506. The axes of the sensor cell 1 are shown in FIG. 10a. The hinge joint 502 is configured to move the linear actuator 503 up and down in the z-direction as well as adjust the angle θ of the linear actuator 503 to the z-axis. The linear actuator is configured to move the indentor 504 to apply a force to the sensor cell 1 with the indentor 504. The rotating platform 506 is configured to rotate the sensor cell 1 to effectively adjust the angle φ of the linear actuator with respect to the x-axis of the sensor cell 1. During the validation study, the hinge joint 502, linear actuator 503, and rotating platform 506 were controlled by the computer 508. Constant load was applied at θ=10°, 20°, 30° and 40°, and φ=90°, 180°, 270° and 360°.

FIG. 10b shows a box and whisker plot of the magnitude of a force vector applied to the sensor cell at θ as calculated using the simulation (y-axis) against the force vector at θ as calculated using the apparatus 50 (x-axis).

FIG. 10c shows a plot of the magnitude of a force vector applied to the sensor cell at φ as calculated using the simulation (y-axis) against the force vector at φ as calculated using the apparatus 50 (x-axis).

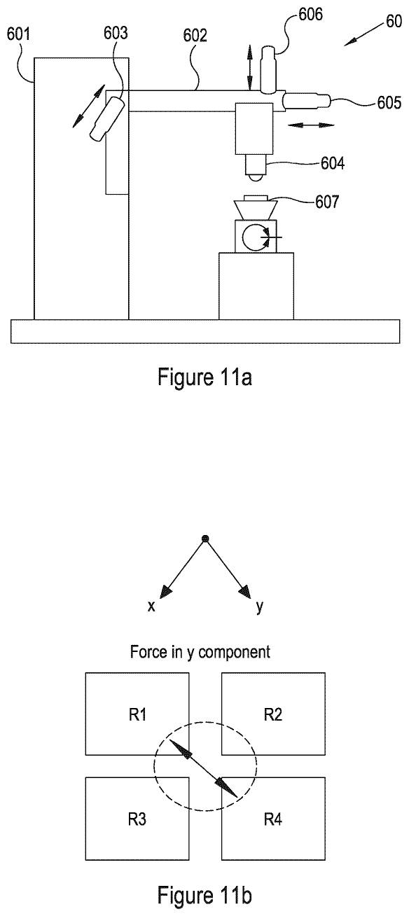

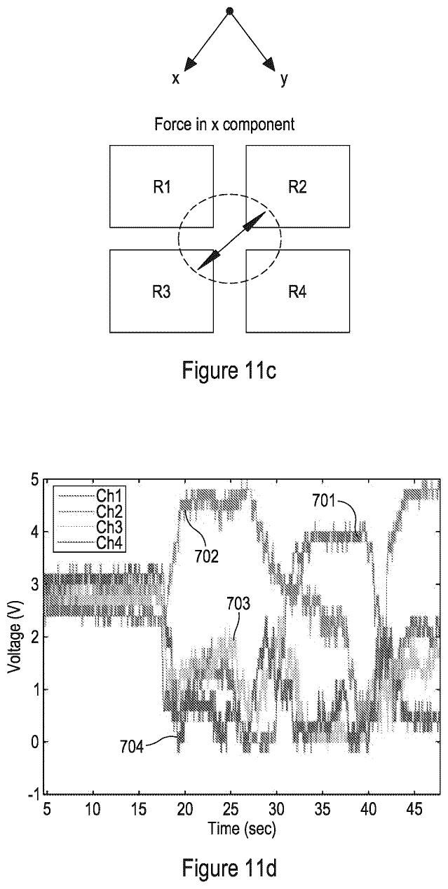

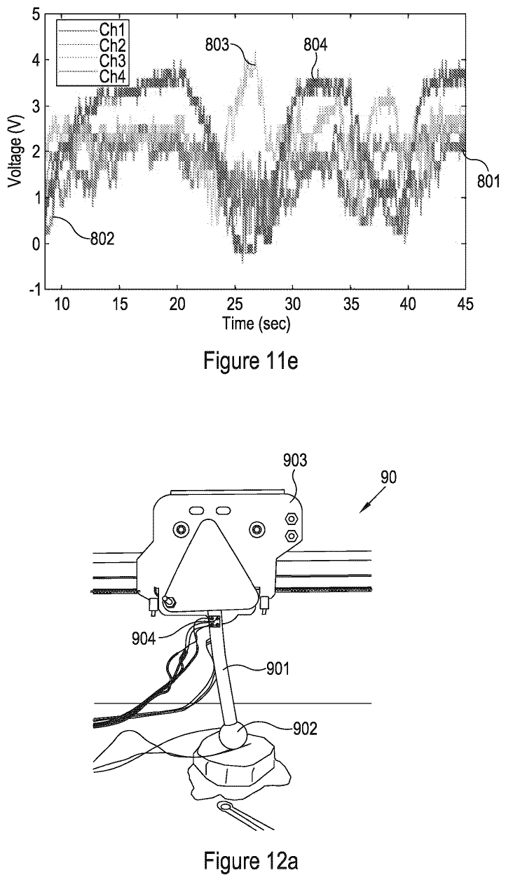

FIG. 11a shows an apparatus 60 used to validate the performance of the sensor cell 1 of FIG. 1 in measuring shear force. The apparatus 60 comprises: a frame 601; an arm 602; a y-axis linear actuator 603; an indentor 604; an x-axis linear actuator 605; a z-axis linear actuator 606; and a rotating platform 607. The arm 602 is coupled to the frame via the y-axis linear actuator 603. The indentor 604 is coupled to the arm via the x-axis linear actuator 605 and the z-axis linear actuator 606. In this way, the indentor 604 can be controlled to move along an x-, y- and z-axis of the apparatus 60.

In an example validation study, the sensor cell 1 was placed on the rotating platform 607 and the rotating platform 607 was rotated to align the axes of the sensor cell 1 with the axes of the apparatus. The sensor cell 1 was set up with the arrangement of reference resistors and voltmeters as shown in FIG. 6. The indentor 604 was controlled to apply an initial normal force to the bump layer of the sensor cell 1, followed by varying shear forces in the positive and negative y-directions, as indicated in FIG. 11b, and in the positive and negative x-directions, as indicated in FIG. 11c.

FIG. 11d shows a graph showing the effect of shear force along the x-axis of the sensor cell 1. The graph shows: voltage measured by the voltmeter arranged to measure voltage across the reference resistor in series with variable resistor R1 (line 701); voltage measured by the voltmeter arranged to measure voltage across the reference resistor in series with variable resistor R2 (line 702); voltage measured by the voltmeter arranged to measure voltage across the reference resistor in series with variable resistor R3 (line 703); and voltage measured by the voltmeter arranged to measure voltage across the reference resistor in series with variable resistor R4 (line 704).

The graph of FIG. 11d shows that between approximately 17s and 31s, there is an increase in the voltage across the reference resistor in series with variable resistor R2, corresponding with a reduction in R2 (line 702) and a decrease in the voltage across the reference resistor in series with variable resistor R3 (line 703) corresponding with an increase in R3. As described above, with reference to FIGS. 4a-j and 6, this indicates a shear force acting on the bump layer of the sensor cell in the negative x-direction.

Similarly, between approximately 31s and 42s, there is an increase in the voltage across the reference resistor in series with variable resistor R1 (line 701) corresponding with a reduction in R1 and a decrease in the voltage across the reference resistor in series with variable resistor R4 (line 704) corresponding with an increase in R4, indicating a shear force acting on the bump layer of the sensor cell in the positive y-direction.

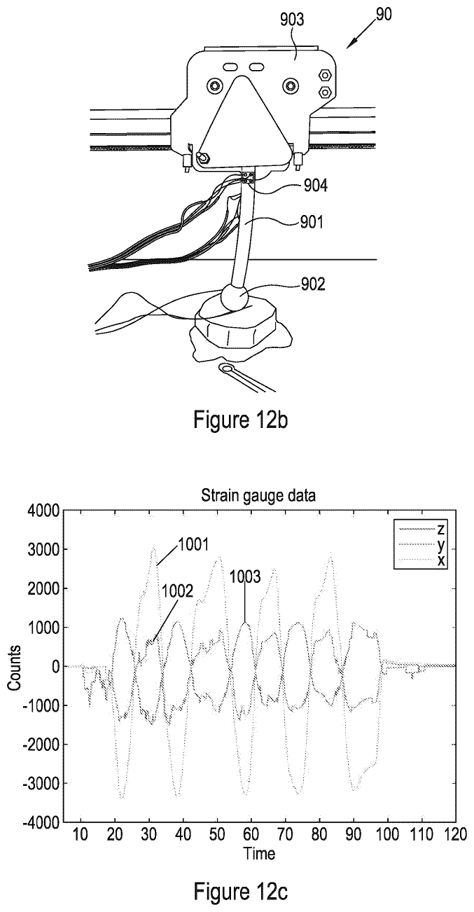

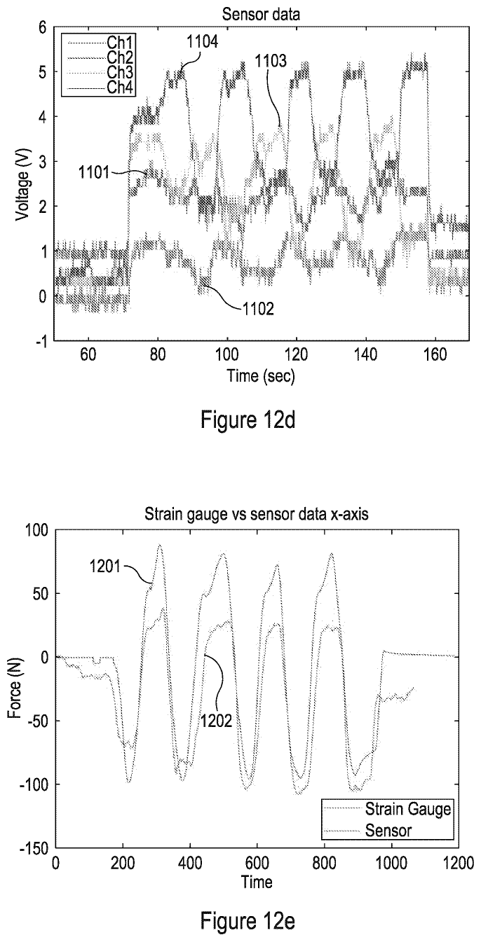

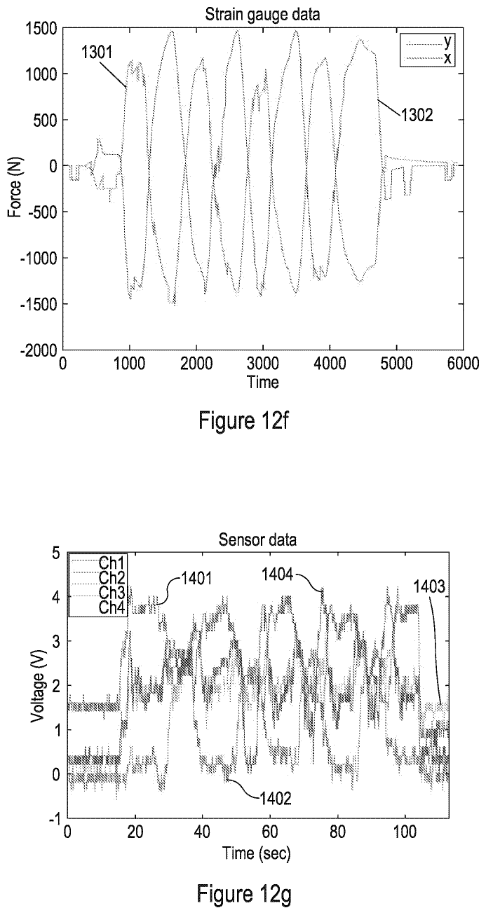

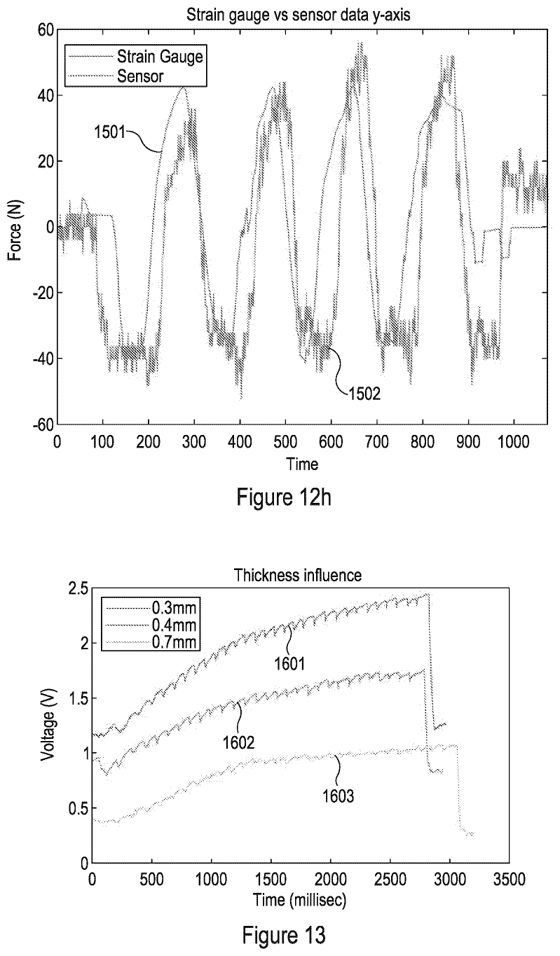

FIG. 11e shows a graph showing the effect of shear force along the y-axis of the sensor cell 1. The graph shows: voltage measured by the voltmeter arranged to measure voltage across the reference resistor in series with variable resistor R1 (line 801); voltage measured by the voltmeter arranged to measure voltage across the reference resistor in series with variable resistor R2 (line 802); voltage measured by the voltmeter arranged to measure voltage across the reference resistor in series with variable resistor R3 (line 803); and voltage measured by the voltmeter arranged to measure voltage across the reference resistor in series with variable resistor R4 (line 804). FIGS. 12a and 12b show another apparatus 90 used to validate the performance of the sensor cell 1 of FIG. 1 in measuring shear force. The apparatus 90 comprises: a beam 901, an indentor 902 coupled to a first end of the beam 901, an actuator 903 coupled to a second end of the beam 901, and a strain gauge arrangement 904 attached to the beam proximate the second end of the beam 901. The actuator 903 is configured to move the indentor 902, via the beam 901, in three axes. The strain gauge arrangement 904 comprises a strain gauge aligned with each of the three axes. In an example validation study, the apparatus 90 was used to apply shear forces, following an initial normal force, to the sensor cell 1 via the indentor 902. The axes of the strain gauges were aligned with x-, y- and z-axes of the sensor cell 1. FIG. 12a shows the apparatus 90 being used to apply a shear force in a first direction and FIG. 12b shows the apparatus 90 being used to apply a shear force in a second direction.

Shear force applied to the sensor cell 1 can be calculated using equation (2) below, based on the theory of simple elastic bending of beams:

$$F = \frac{ε EI}{dy} \qquad ( 2 )$$

Where F is shear force, d is the length of the beam 901, ε is strain as measured by the appropriate strain gauge of the strain gauge arrangement 904, E is the Young's modulus of the beam 901, I is the second moment of area of the beam 901, and y is the distance from the neutral axis (at which the strain is measured).

In the validation study, the sensor cell 1 was set up with the arrangement of reference resistors and voltmeters as shown in FIG. 6. In this way, the sensor cell 1 can be calibrated by mapping a shear force applied to the sensor cell 1, calculated using the equation (2), and the voltage measurements obtained using the voltmeters.

FIG. 12c shows raw data from the strain gauges of the strain gauge arrangement 904 during the application of shear force to the sensor cell along the x-axis. The figure shows strain as measured by the strain gauge arranged to measure strain along the x-axes of the sensor cell (line 1001), strain as measured by the strain gauge arranged to measure strain along the y-axes of the sensor cell (line 1002), and strain as measured by the strain gauge arranged to measure strain along the z-axes of the sensor cell (line 1003).

FIG. 12d shows raw data from the voltmeters during the application of shear force to the sensor cell along the x-axis. The figure shows: voltage measured by the voltmeter arranged to measure voltage across the reference resistor in series with variable resistor R1 (line 1101); voltage measured by the voltmeter arranged to measure voltage across the reference resistor in series with variable resistor R2 (line 1102); voltage measured by the voltmeter arranged to measure voltage across the reference resistor in series with variable resistor R3 (line 1103); and voltage measured by the voltmeter arranged to measure voltage across the reference resistor in series with variable resistor R4 (line 1104).

FIG. 12e shows shear force along the x-axis of the sensor cell 1: as calculated using the strain gauge arrangement 204 and equation (2) (line 1201); and as calculated using the sensor cell 1 following calibration (line 1202).

FIG. 12f shows raw data from the strain gauges of the strain gauge arrangement 904 during the application of shear force to the sensor cell along the y-axis. The figure shows strain as measured by the strain gauge arranged to measure strain along the x-axes of the sensor cell (line 1301) and strain as measured by the strain gauge arranged to measure strain along the y-axes of the sensor cell (1302).

FIG. 12g shows raw data from the voltmeters during the application of shear force to the sensor cell along the y-axis. The figure shows: voltage measured by the voltmeter arranged to measure voltage across the reference resistor in series with variable resistor R1 (line 1401); voltage measured by the voltmeter arranged to measure voltage across the reference resistor in series with variable resistor R2 (line 1402); voltage measured by the voltmeter arranged to measure voltage across the reference resistor in series with variable resistor R3 (line 1403); and voltage measured by the voltmeter arranged to measure voltage across the reference resistor in series with variable resistor R4 (line 1404).

FIG. 12h shows shear force along the y-axis of the sensor cell 1: as calculated using the strain gauge arrangement 204 and equation (2) (line 1501); and as calculated using the sensor cell 1 following calibration (line 1502).

FIG. 13 illustrates how the sensitivity of the sensor cell 1 of FIG. 1 can be adjusted by adjusting the thickness of the substrate (see FIG. 2b). FIG. 13 shows the results of a study of three sensor cells: one having a thickness of 0.3 mm; one having a thickness of 0.4 mm; and one having a thickness of 0.7 mm. Each sensor cell was set up with the reference resistor and voltmeter as shown in FIG. 9d. The power source 101 was arranged to supply a 0.3 A current at 5V across the reference resistor 104 and variable resistors R1, R2, R3, R4 of the sensor cell 1. A stepwise normal load ranging from ON to 60N in increments of 2N was applied across the entire bump layer of each sensor cell.

FIG. 13 shows a graph showing the voltage measured by the voltmeter at each load step for the 0.3 mm sensor cell (line 1601), the 0.4 mm sensor cell (line 1602), and the 0.7 mm sensor cell (line 1603). This graph demonstrates that thicker sensor cells are more sensitive to higher normal loads.

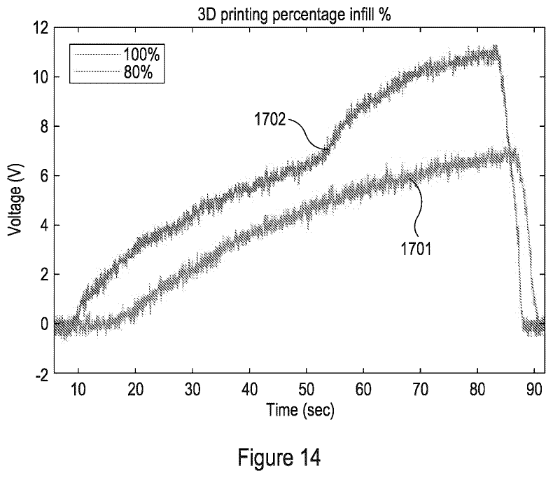

FIG. 14 illustrates how the sensitivity of the sensor cell 1 of FIG. 1 can be adjusted by adjusting the percentage infill of the substrate during manufacture. FIG. 14 shows the result of a study of two sensor cells: one comprising a substrate having a 100% infill; and one comprising a substrate having an 80% infill. Each sensor cell was set up with the reference resistor and voltmeter as shown in FIG. 9d. The power source 101 was arranged to supply a 0.3 A current at 5V across the reference resistor 104 and variable resistors R1, R2, R3, R4 of the sensor cell 1. A stepwise normal load ranging from 0N to 60N in increments of 2N was applied across the entire bump layer of each sensor cell.

FIG. 14 shows a graph showing the voltage measured by the voltmeter at each load step for the 80% infill cell (line 1701) and the 100% infill cell (line 1702). This graph demonstrates that sensor cells with substrated having a lower percentage infill have a lower sensitivity to normal forces.

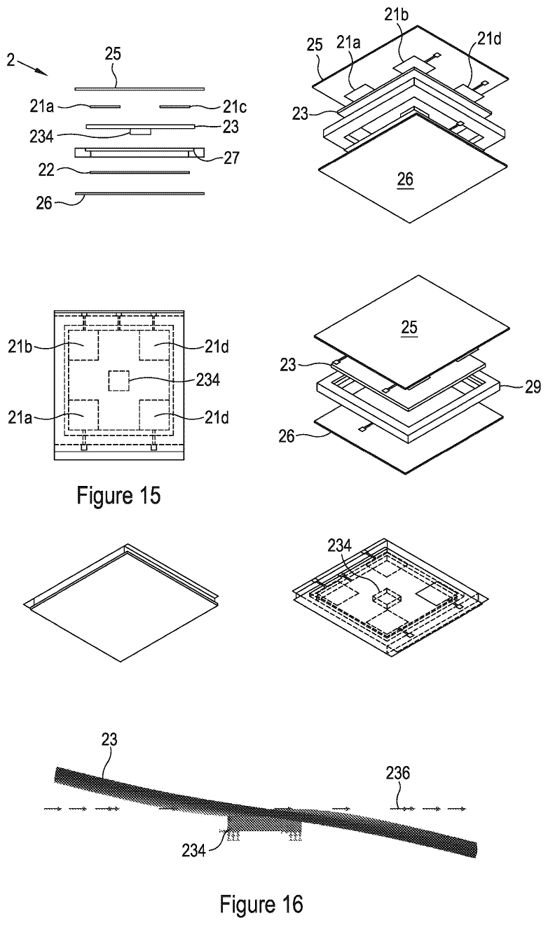

FIG. 15 shows a sensor cell 2 according to a second example embodiment which has many features in common with the sensor cell 1 of FIGS. 1a to 3e (and the features described above, in general, are equally applicable to this, and subsequent embodiments). Reference numerals in FIG. 15 generally correspond with those in FIGS. 1a to 3e, with like features given like numbers, except starting with ‘2’ instead of ‘1’.

In this embodiment, the substrate 23 comprises a square element comprising a flat layer with a central bump 231 attached to the lower surface of the flat layer.

In this example embodiment, the spacer 27 is co-formed with a packaging frame 29 that surrounds the device. The packaging frame 29, together with the the lower insulation layer 26 and the upper insulation layer 25, forms a closed volume within which the electrodes 21a-d, 22 and substrate 23 are disposed (i.e. packaged).

In this embodiment the packaging frame 29, spacer 27 and the substrate 23 are formed using fused filament fabrication. A dual extrusion method may be used to enable both conducting materials (the substrate 23) and non-conducting materials (frame 29, spacer 27) to be printed in a single session. Manufacturing both the substrate and the packaging/spacer as a single part in this way makes packaging of the device very simple. Furthermore, correct alignment between the different portions of the device is simplified.

The sensor cell 2 was tested under uniform pressure in the z direction, and was found to have a sensitivity of 250 Pa/mV and linearity of 0.9 (R² value relative to linear) over a pressure range of 250 kPa to 700 kPa.

Pressure applied to the substrate in the z direction (see FIG. 3a) will deform the substrate 23 down at the peripheral edge (distal to the central spacer). Shear forces will deform the substrate 23 downwards on the side the shear vector points towards, and upward on the side the shear vector points away from. FIG. 16 shows a finite element simulation of the substrate under a shear load, indicated by shear vectors 236 (from left to right), showing the right side of the substrate 23 deformed downwards and the left side of the substrate 23 deformed upwards.

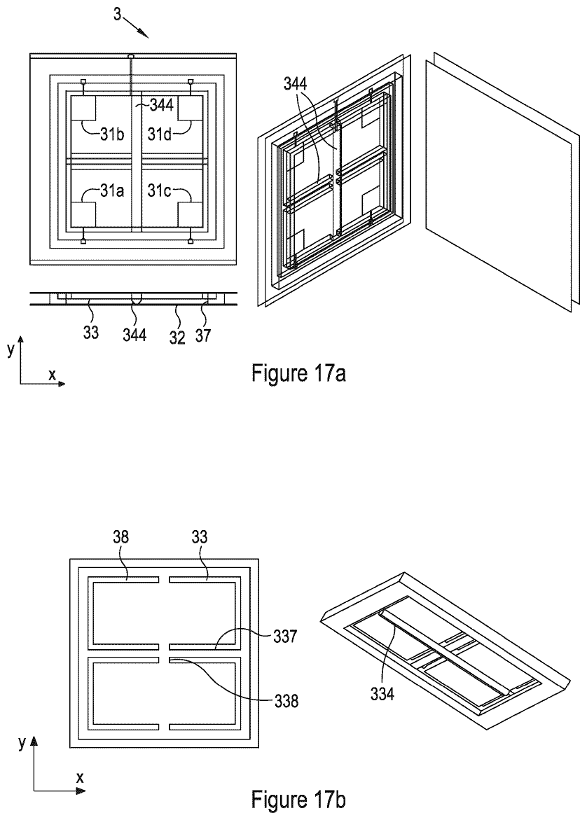

FIGS. 17a and 17b show a sensor cell 3 according to a third example embodiment which also has many features in common with the sensor cell 1 of FIGS. 1a to 3e (and the features described with above are also genearlly applicable to this embodiment). Similarly, features such as the packaging frame, described with reference to the sensor cell 2 are equally applicable in this embodiment. Reference numerals are numbered consistently with those of other embodiments, except starting with ‘3’ instead of ‘1’ or ‘2’.

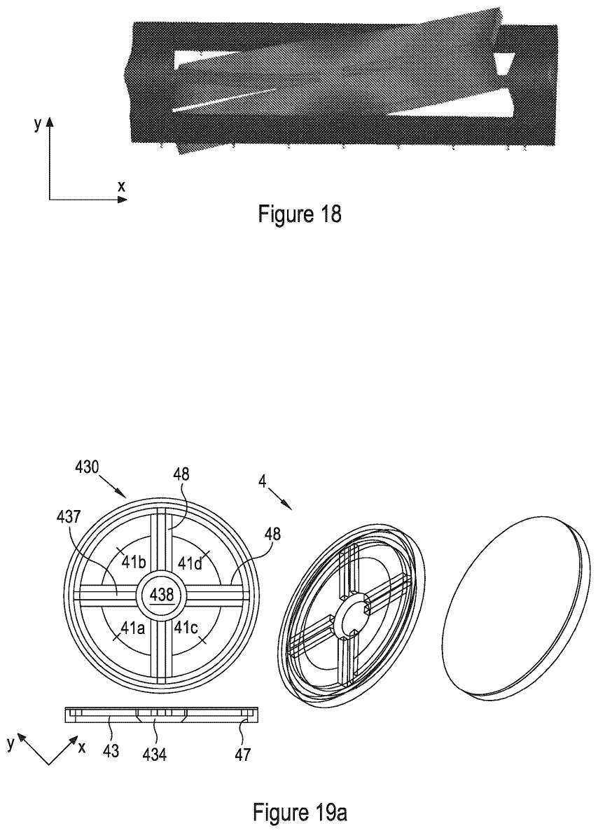

In this embodiment, the substrate 33 comprises a single part 330 which again comprises a flat layer. A line shaped bump 334 is attached in the lower surface of the flat layer, running from edge to edge of the substrate 33 in the y direction. The substrate 33 is patterned with slots 38 which divide the square substrate 33 into two rectangular suspended portions. Each suspended rectangular portion is suspended by connections that bridge the slots 38 in the y direction along the same line that the bump 334 is defined. A central connection 338 is attached to two elongate arms 337 that extend in the x-direction. FIG. 18 shows a finite element simulation of the sensor cell 3 under a shear load in the negative x direction. The short edges of the rectangular portions that the shear vector points toward deform downwards and the short edges of the rectangular portions of that the shear vector points away from deform upwards. Sensor cell 3 is relatively insensitive to shear forces in the y axis (in that differential deformation between the different electrodes is much smaller).

This sensor has been tested, and was found to have two linear response regions under uniform pressure in the z direction: form 0 to 600 kPa at 170 Pa/mV (R² 0.97) and from 600 kPa to 1400 kPa at 260 Pa/mV (R² 0.98).

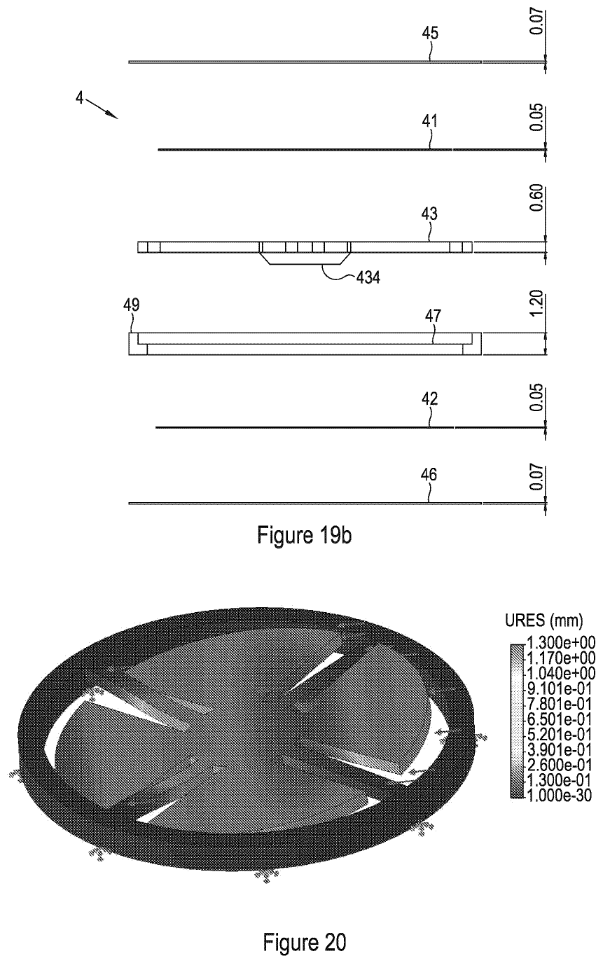

FIGS. 19a and 19b show a sensor cell 4 according to a fourth example embodiment which also has many features in common with the sensor cell 1 of FIGS. 1a to 3e, and also features in common with the sensor cell 2 and sensor cell 3 (and the features described with reference to the previous embodiments are equally applicable to this embodiment). Reference numerals in FIG. 19 are numbered consistently with those of other embodiments, except starting with ‘4’ instead of ‘1’ or ‘2’ or ‘3’.

In this embodiment, the substrate 43 comprises a single circular part 430 which again comprises a flat layer. A circular shaped central bump 434 is attached in the lower surface of the flat layer, to a corresponding circular boss 438 of the flat layer. The substrate 43 is patterned with slots 48 which divide the circular substrate 43 into four quadrants. Each of the quadratns is separated from the circumferentially adjacent quadrant by a radial suspension arm 437 (also defined by the slots 48). Each of the four radial suspension arms run from an edge of the substrate (which will be supported by the spacer) to the circular boss 438 to which the bump 434 is fixed.

The upper electrodes 41a-d are annular, and disposed on the outer edge of each quadrant (or petals) of the sensor cell 4. In the example of FIG. 19, the radial suspension arms are each at 45 degrees to the x and y directions. The sensor cell has fourth order rotational symmetry about the central bump.

In this example embodiment, the spacer 47 is co-formed with a packaging frame that surrounds the device. The packaging frame, together with the the lower insulation layer and the upper insulation layer, forms a closed volume within which the electrodes 41a-d, and substrate 43 are disposed (i.e. packaged).

FIG. 19b shows representative thicknesses for each of: the top insulation layer (which may be polyimide), 0.07 mm; top electrodes (which may be copper), 0.05 mm; substrate (which may be carbon black loaded thermoplastic), 0.6 mm, with a 0.6 mm thick bump; packaging frame 49, 1.2 mm comprising spacer 47, 0.6 mm (co-formed from non-conductive thermoplastic); lower electrode 42 (e.g. copper), 0.05 mm; lower insulating layer 46 (e.g. polyimide) 0.07 mm. Other layer thicknesses may be used—these are merely illustrative.

FIG. 20 shows a finite element simulation of the sensor cell 4 under a shear load in the negative x direction. The quadrant/petal that extends in the negative x direction is deformed downwards, and the quadrant/petal that extends in the positive x direction is deformed upwards. Due to the symmetry of the design, it can be readily understood that shear loads in the opposite direction, or in the positive or negative y direction will similarly deform different quadrants/petals up or down. It will also be understood that this sensor will function with a mechanism similar to that described with reference to FIGS. 4a to 4j.

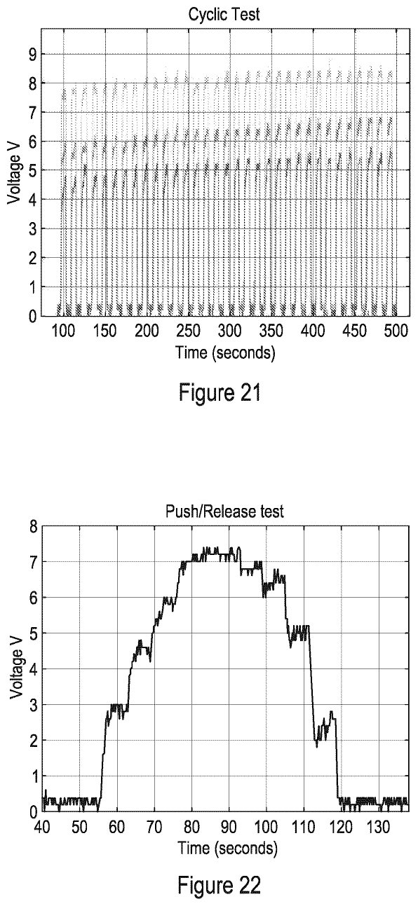

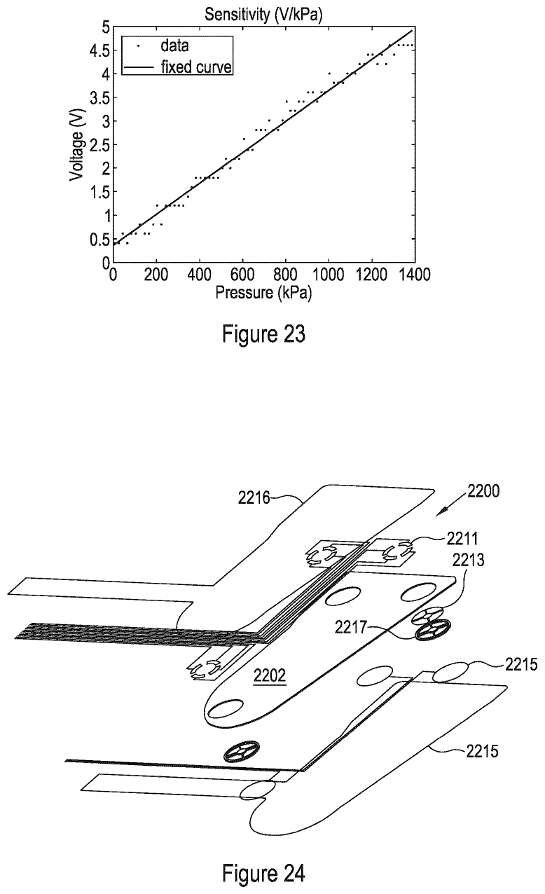

FIG. 21 shows the results of cyclic testing of 900 kPa of uniform pressure in the z direction. The output of the sensor cell 4 remains consistent. FIG. 22 shows a push/release test, in which a uniform z pressure was varied in 50 kPa increments. There is very little hysteresis according to this embodiment (<5%). FIG. 23 shows sensitivity and linearity of the sensor cell 4, which is substantially linear over the range 0 kPa to 1400 kPa.

Sensor devices according to embodiments may be used in a wide range of applications. While one application is in shoes, embodiments may also help measure shear forces between any adjacent surfaces where a small, low-profile sensor is needed. Sensor cell 2 according to the second example embodiment and similar devices may be particularly suitable for use in wearables, prosthetics, surgical robot haptics, navigate a virtual player using the shear force feedback and the same time control its speed using the normal force feedback, and artificial pressure-sensitive skins in the healthcare sector providing high resolution pressure mapping. Sensor cell 4 according to the fourth example embodiment 4 and similar devices may be used in industry, such as a load cell of triaxial mechanical test machine. Another application could be for the fine tuning of grasping motor control of industrial robotic arms. A third sector which could benefit is in sports, for example, collecting data about football players to analyse their training performance in terms of gait analysis or could form a tactile patch attached to the shoe for analysing shooting accuracy and pressure mapping between the foot and the ball. Further applications exist in entertainment, where sensor cells according to embodiments may be used in gaming controllers and TV remote controllers, for example to navigate a virtual player or a cursor in on a screen, allowing a low profile and lighter device rather the use of bulky joysticks. Adjustments to the design can be made to fit the requirements of each of these different applications. Such fine-tuning may include altering the percentage infill of 3d printing of the substrate materials or the thickness of material layers.

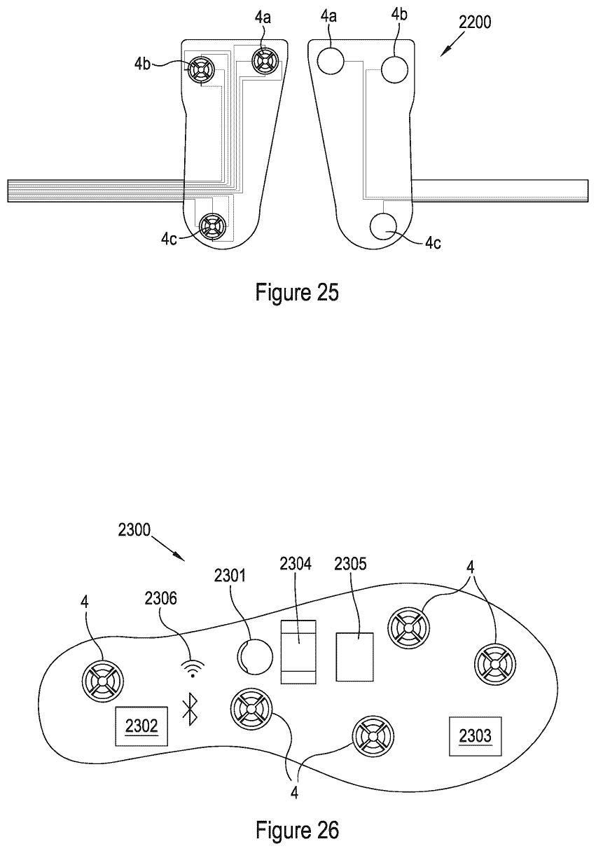

FIGS. 24 and 25 show an insole sensor 2200 according to an embodiment. FIG. 23 shows top and bottom views of the device, and FIG. 22 is an exploded diagram showing the different layers of the sensor 2220. The insole sensor 2200 comprises a flexible printed circuit board, for example comprising one or more polyimide insulating layers (e.g. Kapton) and one or more conducting copper layers. Using traces in a flexible printed circuit board is advantageous over connecting discrete sensors with wires, because the traces are better supported by the flexible PCB, thereby minimising disturbances to signals as a result of motion while walking.

The insole sensor 2200 comprises: a lower insulating substrate (e.g. Kapton) 2215, upper insulating substrate 2216, lower electrodes 2212, upper electrodes 2211, sensor spacer 2217, insole spacer 2202, and sensor substrate 2213.

The insole sensor 2200 comprises sensor cells 4a-c similar to that shown in FIG. 19, positioned in different high pressure regions of interaction between the foot and the insole (which may be determined for a specific user, for example based on a wear pattern of a used insole). The positions of the sensor cells 4a-c are configured to correspond approximately with the positions shown in FIG. 7.

The upper electrodes of the sensor cells 4a are implemented in the upper conducting layer of the flexible PCB, which also defines conducting traces connecting the electrodes to a connection region at the edge of the printed circuit board. The lower electrodes of the sensor cells 4a are implemented in the lower conducting layer of the flexible PCB, which also defines conducting traces connecting the electrodes to a connection region at the edge of the printed circuit board.

An electronic circuit may be configured to monitor the sensors as a subject walks while wearing a shoe that comprises the insole sensor 2200. A centre of pressure may be approximated, based on the output of the sensors. The x and y locations of the centre of pressure may be calculated using the below equations. In these equations, x_{A}, x_{B} & x_{C} are the locations of each sensor with respect to the centre of the sole and along the x-axis, and force_{xA}, force_{xB} & force_{xC} are the forces of each one. Similarly, y_{A}, y_{B}, y_{C}, force_{yA}, force_{yB} & force_{yC} are the locations and forces along the y-axis.

x_{center}=(x_{A}*|force_{xA}|)+(x_{B}*|force_{xB}|)+(x_{C}*|force_{xC}|)/|force_{xA}|+|force_{xB}|+|force_{xC}|

y_{center}=(y_{A}*|force_{yA}|)+(y_{B}*|force_{yB}|)+(y_{C}*|force_{yC}|)/|force_{yA}|+|force_{yB}|+|force_{yC}|

The resultant force at the centre-of-pressure point (x_{center}, y_{center}) may be calculated using the following equations.

Fₓ_{_{center}}=force_{xA}+force_{xB}+force_{xC}

F_{y}_{_{center}}=force_{yA}+force_{yB}+force_{yC}

F_{z}_{_{center}}=force_{zA}+force_{zB}+force_{zC}

The insole depicted in FIGS. 24 to 25 was tested with a treadmill comprising a calibrated force plate. Good correlation was observed between the estimated centre of pressure determined from the output of the insole according to an embodiment and the results from the calibrated force plate, during: slow walking, fast walking and slow running. Correlation coefficients for the force vector were between 0.8 and 0.88 and for the centre of pressure location, between 0.72 and 0.86.

Referring to FIG. 26, an example insole system 2300 is shown comprising: sensors 4, battery 2301, microcontroller/microprocessor 2304, multiplexer 2305, at least one inertial measurement unit 2302, 2303 and wireless communication device 2306. The multiplexer 2305 is configured to receive signals from the sensors 4, and to provide the multiplexed signals to the microcontroller 2304. The microcontroller 2304 is configured to store and/or process the signals, for example to determine force vectors and/or centres of pressure, or to communicate signals and/or measurements derived from the signals via the wireless communication device (for example to a cloud based server, optionally via a mobile device such as a smartphone). The intertial measurement units 2032, 2303 provide orientation and motion data, which may be combined with the force/pressure measurements from the sensors 4 by the microcontroller 2304.

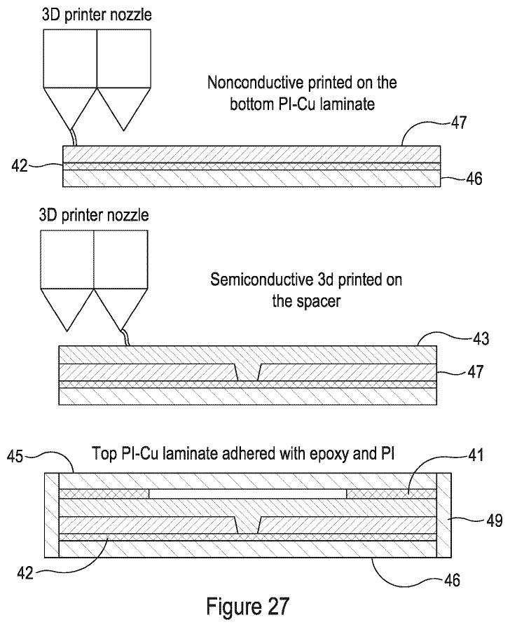

FIG. 27 illustrates a method of making an example sensor. The reference numerals given correspond with those given in respect of the embodiment of FIGS. 19a and 19b, but it will be understood that this manufacturing approach is applicable to any embodiment. An additive manfuacturing tool comprising a first extrusion nozzle and a second extrusion nozzle may be used. The first extrusion nozzle may be arranged to extrude a filament a first material (e.g. a non-conductive thermoplastic) and the second extrusion nozzle may be configured to extrude a filament of a second material (e.g. a conducting thermoplastic, which may be loaded with carbon black). A single manufacturing session may be used to form conducting and non-conducting portions of a sensor cell using the first and second extrusion nozzles. The spacer 47 (and packaging frame, not shown) and flexible substrate 43 may be formed on a flexible PCB comprising the lower insulating layer 46 and lower electrodes 42. A further flexible PCB comprising the upper electrodes 41 and upper insulating layer 45 is laminated onto the packaging frame to form the sensor cell.

Although specific embodiments have been described, these examples are not intended to limit the scope of the invention, which should be determined with reference to the appended claims.

1-28. (canceled)

29. A sensor cell for measuring force, comprising:
a conductive elastic substrate; and
a variable resistor comprising:
a first electrode;
a second electrode; and
a portion of the conductive elastic substrate arranged between the electrodes such that a force acting on the substrate causes a change in distance between the electrodes thereby changing a resistance of the variable resistor.

30. The sensor cell of claim 29, comprising:
a plurality of variable resistors each comprising:
a first electrode;
a second electrode; and
a portion of the conductive elastic substrate, wherein the portion of the substrate is arranged between the electrodes; wherein
a shear force acting on the substrate causes a distance between the electrodes of a first subset of the resistors to increase and a distance between the electrodes of a second subset of the resistors to decrease, and a pressure acting on the substrate causes a distance between the electrodes of both subsets of resistors to decrease.

31. The sensor cell of claim 30, wherein each subset comprises two resistors, wherein:
a shear force acting on the substrate along a first axis in a first direction causes a distance between the electrodes of a first resistor of the first subset of resistors to decrease and a distance between the electrodes of a second resistor of the first subset of resistors to increase;
a shear force acting on the substrate along the first axis in a second direction opposite the first direction causes a distance between the electrodes of the first resistor of the first subset of resistors to increase and a distance between the electrodes of the second resistor of the first subset of resistors to decrease;
a shear force acting on the substrate along a second axis in a first direction causes a distance between the electrodes of a first resistor of the second subset of resistors to decrease and a distance between the electrodes of a second resistor of the second subset of resistors to increase; and
a shear force acting on the substrate along the second axis in a second direction opposite the first direction causes a distance between the electrodes of the first resistor of the second subset of resistors to increase and a distance between the electrodes of the second resistor of the second subset of resistors to decrease.

32. The sensor cell of claim 30, wherein the first electrodes of the variable resistors are separate, and the variable resistors share a common second electrode.

33. The sensor cell of claim 29, wherein the substrate comprises petals or lobes separated by slots and/or wherein the substrate is suspended by radial suspension arms between the petals that extend from an edge of the substrate to a central boss region of the substrate.

34. The sensor cell of claim 29, wherein the substrate comprises a first part and a second part, each part comprising:
a first portion and a second portion;
an inclined portion extending from the first portion to the second portion;
a pivot arranged on the first portion;
a slot extending inwards from an edge of the inclined portion and extending perpendicularly to the pivot; wherein
the first and second parts are connected together at the respective slots.

35. The sensor cell of claim 29, wherein at least a part of the substrate is additive manufactured and/or wherein at least a part of the substrate comprises thermoplastic polyurethane with carbon black.

36. The sensor cell of claim 29, comprising an insulation layer, wherein the first electrode of each variable resistor is arranged between the insulation layer and the substrate.

37. The sensor cell of claim 36, wherein the sensor cell comprises one or more bumps coupled to the substrate such that the or each bump exerts a force on the substrate when a corresponding force is exerted on the bump, and wherein the insulation layer is arranged between the bump and the first electrode of each variable resistor.

38. The sensor cell of claim 29, comprising an insulation layer, wherein the second electrode of each variable resistor is arranged between the insulation layer and the substrate.

39. The sensor cell of claim 29, comprising a spacer arranged between the substrate and the second electrode of each variable resistor, wherein the spacer comprises an open area to allow contact between the substrate and the respective electrode.

40. The sensor cell of claim 39, wherein the spacer comprises polydimethylsiloxane or a non-conducting thermoplastic and/or wherein the sensor cell further comprises a packaging frame that is co-formed with the spacer.

41. A system for measuring force, comprising:
the sensor cell of claim 29;
a power source arranged to provide a potential difference between the first electrode and the second electrode of each variable resistor;
one or more measuring devices configured to measure a change in an electrical property resulting in a change of resistance of each variable resistor caused by a force acting on the substrate; and
a processor configured to determine the magnitude of the force acting on the substrate in dependence on the change in the electrical property.

42. The system of claim 41, comprising a reference resistor of fixed resistance for each variable resistor, wherein each reference resistor is connected in series with one of the variable resistors, wherein the one or more measuring devices comprises a voltmeter for each reference resistor, wherein each voltmeter is arranged to measure voltage across one of the reference resistors, and wherein the electrical property is voltage across each reference resistor.

43. A method of manufacturing a sensor cell, the sensor cell comprising:
a conductive elastic substrate;
a plurality of variable resistors each comprising:
a first electrode;
a second electrode, wherein the first electrode of each variable resistor is separate, and the resistors share a common second electrode; and
a portion of the conductive elastic substrate, wherein the portion of the substrate is arranged between the electrodes;
an insulation layer, wherein the second electrode is arranged between the insulation layer and the substrate; wherein
a shear force acting on the substrate causes a distance between the electrodes of a first subset of the resistors to increase and a distance between the electrodes of a second subset of the resistors to decrease, and a pressure acting on the substrate causes a distance between the electrodes of both subsets of resistors to decrease;
the method comprising:
forming the first electrodes on the insulation layer using an etching process;
adhering the substrate to the first electrodes; and
adhering the second electrode to the substrate.

44. The method of claim 43, comprising forming at least part of the substrate using an additive manufacturing process.

45. The method of claim 44, comprising adjusting a thickness of the substrate and/or adjusting a percentage printing infill of the substrate in dependence on a required sensitivity of the sensor cell.

46. The method of claim 43, wherein the sensor cell comprises a spacer arranged between the substrate and the second electrode, wherein the spacer comprises an open area to allow contact between the substrate and the second electrode, and wherein the method comprises forming the spacer by an additive manufacturing process.

47. The method of claim 46, wherein the substrate and spacer are formed in a single additive manufacturing session.

48. The method of claim 47, further comprising forming a packaging frame for the sensor cell that is co-formed with the spacer.
